(19) United States
(12) Patent Application Publication
(10) Pub. No.: US 2024/0101972 A1
(43) Pub. Date: Mar. 28, 2024

(54) METHODS OF REGULATING ADENO-ASSOCIATED VIRUS PRODUCTION

(71) Applicant: SPARK THERAPEUTICS, INC., Philadelphia, PA (US)

(72) Inventors: Mustafa Yazicioglu, Broomall, PA (US); Ahmet Yunus Ozdemir, Voorhees, NJ (US)

(73) Assignee: SPARK THERAPEUTICS, INC., Philadelphia, PA (US)

(21) Appl. No.: 18/535,857

(22) Filed: Dec. 11, 2023

Related U.S. Application Data
(63) Continuation of application No. PCT/US22/33071, filed on Jun. 10, 2022.
(60) Provisional application No. 63/209,735, filed on Jun. 11, 2021.
(60) Provisional application No. 63/350,849, filed on Jun. 9, 2022.

(51) Int. Cl. C12N 7/00; C07K 14/005; C12N 9/06

(52) U.S. Cl. CPC C12N 7/00; C07K 14/005; C12N 9/003; C12Y 105/01003; C07K 2319/95; C12N 2750/14122; C12N 2750/14143; C12N 2750/14152

(57) ABSTRACT

The presently disclosed subject matter relates to compositions and methods for regulating recombinant adeno-associated virus (rAAV) production in cell culture. In particular, the presently disclosed subject matter relates to strategies to overcome AAV Rep protein-mediated cytotoxicity by reversible post-translational regulation of the expression of AAV Rep and helper proteins, resulting in regulated rAAV production.

### CROSS-REFERENCE TO RELATED APPLICATIONS

This application is a continuation of International Patent Application No. PCT/US2022/033071 filed Jun. 10, 2022, which claims priority to U.S. Provisional Application No. 63/350,849, filed on Jun. 9, 2022, and to U.S. Provisional Application No. 63/209,735, filed on Jun. 11, 2021, both of which are hereby incorporated by reference in their entireties, and to which priority is claimed.

### SEQUENCE LISTING

The instant application contains a Sequence Listing which has been submitted electronically in XML format and is hereby incorporated by reference in its entirety. Said sequence listing copy, created on Dec. 11, 2023, is named 089504.0129SL.xml and is 29, 849 bytes in size.

### 1. FIELD OF INVENTION

The presently disclosed subject matter relates to compositions and methods for the regulation of recombinant adeno-associated virus (rAAV) production in cell culture. In particular, the presently disclosed subject matter relates to strategies to overcome AAV Rep protein-mediated cytotoxicity by reversible post-translational regulation of the expression of AAV Rep protein, resulting in regulated rAAV production.

### 2. BACKGROUND

There are a variety of AAV production systems used to produce rAAV in cell culture. These include plasmid transient transfection of human embryonic kidney (HEK) 293 cells, Hela producer cell lines, BHK21-based platforms, and baculovirus-based production systems. Each of these systems has strengths and weaknesses. For example, given the importance of the adenovirus Ela protein in initiating the production of rAAV, E1a-expressing cells, e.g., HEK293 cells, are attractive to produce rAAV as they eliminate the need to otherwise introduce an Ela gene into the host cell genome. E1a-expressing cells, e.g., HEK293 cells, can also offer ease of growth and adaptability to growth in suspension. However, efforts to create stable and passagable rAAV producing E1a-expressing cell lines have been hampered by cellular toxicity caused by E1a-induced accumulation of AAV Rep protein. In view of the foregoing, there is a need in the art for new rAAV production strategies where accumulation of Rep protein can be regulated to avoid Rep-mediated cytotoxicity, resulting in regulated rAAV production.

### 3. SUMMARY OF THE INVENTION

In certain embodiments, the present disclosure is directed to methods of regulating the production of recombinant adeno-associated virus (rAAV) vector particles, the method comprising: introducing into a cell: an rAAV comprising a gene of interest and a nucleic acid encoding a fusion protein, wherein the fusion protein comprises an AAV protein, and a degradation ligand-dependent degradation domain, culturing the cell under conditions suitable for producing the rAAV vector particles; and contacting the cell with a degradation ligand, wherein the degradation ligand binds to the degradation domain to regulate the expression of the AAV protein and thereby regulate the production of rAAV vector particles.

In certain embodiments, the nucleic acid encoding a fusion protein comprises the Rep protein, a linker, and a degradation ligand-dependent degradation domain. In certain embodiments, the ligand-dependent degradation domain is derived from FKBP. In certain embodiments, the degradation ligand-dependent degradation domain is DHFR. In certain embodiments, the degradation ligand-dependent degradation domain is an auxin induced degradation helper domain. In certain embodiments, the degradation ligand is a small molecule ligand. In certain embodiments, the small molecule is Shield1. In certain embodiments, the small molecule is TMP. In certain embodiments, the small molecule is auxin. In certain embodiments the small molecule is dTag13.

In certain embodiments, the nucleic acid encoding a fusion protein comprises the Cap protein, a linker, and a degradation ligand-dependent degradation domain. In certain embodiments, the ligand-dependent degradation domain is derived from FKBP. In certain embodiments, the degradation ligand-dependent degradation domain is DHFR. In certain embodiments, the degradation ligand-dependent degradation domain is an auxin induced degradation domain. In certain embodiments, the degradation ligand is a small molecule ligand. In certain embodiments, the small molecule is Shield1. In certain embodiments, the small molecule is TMP. In certain embodiments, the small molecule is auxin, In certain embodiments the small molecule is dTag13.

In certain embodiments, the nucleic acid encoding a fusion protein comprises a Helper protein, a linker, and a degradation ligand-dependent degradation domain. In certain embodiments, the Helper protein is E2. In certain embodiments, the ligand-dependent degradation domain is derived from FKBP. the degradation ligand-dependent degradation domain is DHFR. In certain embodiments, the degradation ligand-dependent degradation domain is an auxin induced degradation domain. In certain embodiments, the degradation ligand is a small molecule ligand. In certain embodiments, the small molecule is Shield1. In certain embodiments, the small molecule is TMP. In certain embodiments, the small molecule is auxin. In certain embodiments the small molecule is dTag13.

In certain embodiments, the cell is an E1a-expressing cell. In certain embodiments, the E1a-expressing cell is a HEK293 cell.

In certain embodiments, the Rep protein is Rep78, Rep68, Rep52, or Rep40 protein.

In certain embodiments, the degradation ligand-dependent degradation domain is fused to the C-terminal end of the AAV protein. In certain embodiments, the degradation ligand-dependent degradation domain is fused to the N-terminal end of the AAV protein.

In certain embodiments, the linker is a flexible linker. In certain embodiments, the linker is a rigid linker.

In certain embodiments, the methods of the present disclosure comprise introducing into the cell a nucleic acid encoding a Cap protein. In certain embodiments, the nucleic acid encoding the fusion protein and the nucleic acid encoding a Cap protein are introduced into the cell using at least one plasmid. In certain embodiments, the nucleic acid encoding the fusion protein and the nucleic acid encoding a Cap protein are introduced into the cell using the same plasmid. In certain embodiments, the nucleic acid encoding the fusion protein and the nucleic acid encoding a Cap protein are introduced into the cell using separate plasmids. In certain embodiments, the AAV fusion protein encoding gene and/or cap gene are under the control of a regulatory element. In certain embodiments, the regulatory element is a promoter. In certain embodiments the regulatory element is a Tet response element.

In certain embodiments, the cell is a eukaryotic cell. In certain embodiments, the eukaryotic cell is an animal cell. In certain embodiments, the animal cell is a mammalian cell. In certain embodiments, mammalian cell is a HEK cell.

In certain embodiments, the present disclosure is directed to an rAAV producing cell, wherein the cell comprises a nucleic acid encoding a fusion protein comprising an AAV protein and a degradation ligand-dependent degradation domain. In certain embodiments, the nucleic acid encoding the fusion protein comprises the AAV protein, a linker, and a degradation ligand-dependent degradation domain. In certain embodiments, the degradation ligand-dependent degradation domain is derived from FKBP. In certain embodiments, the ligand-dependent degradation domain is derived from FKBP. In certain embodiments, the degradation ligand-dependent degradation domain is DHFR. In certain embodiments, the degradation ligand-dependent degradation domain is an auxin induced degradation domain. In certain embodiments, the degradation ligand is a small molecule ligand. In certain embodiments, the small molecule is Shield1. In certain embodiments, the small molecule is TMP. In certain embodiments, the small molecule is auxin. In certain embodiments the small molecule is dTag13.

In certain embodiments, the rAAV producing cell is a eukaryotic cell. In certain embodiments, the eukaryotic cell is an animal cell. In certain embodiments, the animal cell is a mammalian cell. In certain embodiments, the mammalian cell is a HEK cell. In certain embodiments, the cell is an E1a-expressing cell. In certain embodiments, the E1a-expressing cell is a HEK293 cell.

In certain embodiments, the rAAV producing cell comprises a ligand-dependent degradation domain fused via the linker to the C-terminal end of the AAV protein. In certain embodiments, the ligand-dependent degradation domain is fused via the linker to the N-terminal end of the AAV protein. In certain embodiments, the linker is a flexible linker. In certain embodiments, the linker is a rigid linker.

In certain embodiments, the cell comprises a nucleic acid encoding a Cap protein. In certain embodiments, the nucleic acid encoding the fusion protein and the nucleic acid encoding a Cap protein are introduced into the cell using at least one plasmid. In certain embodiments, the nucleic acid encoding the fusion protein and the nucleic acid encoding a Cap protein are introduced into the cell using the same plasmid. In certain embodiments, the nucleic acid encoding the fusion protein and the nucleic acid encoding a Cap protein are introduced into the cell using separate plasmids. In certain embodiments, the AAV fusion protein encoding gene and/or cap gene are under the control of a regulatory element. In certain embodiments, the regulatory element is a promoter. In certain embodiments the regulatory element is a Tet response element.

### 4. BRIEF DESCRIPTION OF THE DRAWINGS

FIG. 1 depicts examples of plasmid constructs finding use in the method and systems of the instant disclosure.

FIG. 2 depicts a schematic for an experimental flow for confirming rAAV production using Degron constructs such as those shown in Figure. 1.

FIG. 3 shows that rAAV can be produced by transfection of mammalian cells having Rep and Cap genes on separate plasmids.

FIG. 4 shows that Rep-Degron constructs can be used for AAV production and the accumulation of Rep protein can be regulated by the addition of the Shield-1 molecule.

FIG. 5 shows the Shield-1-mediated post-translational regulation of expression of Rep constructs where the construct encodes Rep with a C-terminal degron fusion or Rep with an N-terminal degron fusion.

FIG. 6 shows Shield-1-mediated post-translational regulation of expression of Rep protein with or without a C-terminal degron fusion, and where the Rep is present on the same construct as Cap or present on a separate construct.

FIG. 7 shows that rAAV production can be modulated by the fusion of a C-terminal degron to Rep and the addition of increasing concentrations of Shield-1; and that increasing concentrations of Rep-degron plasmid where the Rep expression is under a CMV promoter results in reduced rAAV production.

FIG. 8 shows rAAV production using Rep protein with or without a C-terminal degron fusion and incorporation of rigid or flexible linkers between Rep and the degron.

FIG. 9 shows rAAV production using Rep protein with or without an N-terminal degron fusion and incorporation of rigid or flexible linkers between Rep and the degron.

FIG. 10 shows regulation with Shield-1 of Rep constructs with C-terminal Degron and of Rep constructs with N-terminal Degron.

FIG. 11 depicts the possible size changes in a western blot after the addition of a degron tag to the N-terminal or C-terminal of Rep protein. Addition of degron to the N-terminal of Rep protein (N-degron) changes the Molecular Weight of the large Rep but not the small Rep protein. Small Rep Molecular Weight is not affected by an N-terminal protein tag because p19 promoter, which drives the expression of small Rep, located within the Rep gene. Small Rep protein does not share the same N-terminal sequence as the large Rep protein. In contrast, addition of a degron to C-terminal of Rep protein (C-degron) changes the Molecular Weights of both small and large Rep proteins as C terminal is same for both. Addition of small molecule such as Shield1 or TMP to the cell culture media can inhibit the protein degradation and the band intensity will change accordingly. Example shown here is FKBP derived degron.

FIGS. 12A-12D depict the regulation of AAV production via Rep proteins with FKBP derived degron. FIG. 12A depicts a western blot of N-terminal FKBP degron tagged Rep proteins. FIG. 12B depicts a western blot of C-terminal FKBP degron tagged Rep proteins. FIG. 12C depicts the AAV titers of the samples from FIGS. 12A and 12B. FIG. 12D depicts the AAV titers with Rep and Cap plasmids transfected at different ratios.

FIGS. 13A-13C depict the regulation of AAV production via Rep proteins with E. coli DHFR derived degron (ecDHFR degron). FIG. 13A depicts a western blot of N-terminal ecDHFR degron tagged Rep proteins. FIG. 13B depicts a western blot of C-terminal ecDHFR degron tagged Rep proteins. FIG. 13C depicts the AAV titers of the samples from FIGS. 13A and 13B.

FIGS. 14A-14B depict the regulation of AAV production via Rep proteins with auxin based degron and ecDHFR degron. FIG. 14A depicts a western blot of Reps that are tagged with auxin Inducible degron or ecDHFR degron. FIG. 14B depicts the AAV titers of the samples from FIG. 14A.

FIG. 15 depicts the impact of different doses of Shield1 and dTag13 on AAV production. dTAG-13 is a small molecule that can target mutant FKBP sequences for ubiquitin mediated degradation. It can function by linking the targeted protein sequence to a E3 ubiquitin ligase, cereblon. dTAG-13 can lead to degradation of FKBP fusion proteins and proteins fused to it.

FIGS. 16A-16B depict a western blot of Rep with C-terminal degron under the control of a Tet response element containing promoter (TRE3G) and exposure to Tet protein and a single doxycycline concentration (FIG. 16A) and the AAV titers of the samples from FIG. 16A (FIG. 16B).

FIGS. 17A-17B depict a western blot of Rep with C-terminal degron under the control of a TRE3G promoter and exposure to Tet protein a range of doxycycline concentrations (FIG. 17A) and the AAV titers of the samples from FIG. 17A (FIG. 17B).

FIGS. 18A-18C depict the effect of a p5 promoter on Rep protein levels in a Tet system, in particular the TRE3G-Rep-Degron system (FIG. 18A); a western blot of Rep constructs with C-terminal degron and TRE3G promoter (FIG. 18B); and the AAV titers of the samples from FIG. 18B (FIG. 18C)

FIGS. 19A-19B. FIG. 19A depicts the DBP protein expression observed from an E2A gene tagged with an FKBP derived degron motif. The plasmid expressing E2A-DBP-degron was transfected along with other plasmids expressing Rep/Cap, ITR-GOI, E4-E34K and VA2. At the end of 72 hours, the cells were lysed and AAV titer levels were analyzed. The cells transfected only with ITR-GOI plasmid (where Helper and Rep/Cap plasmids were omitted to prevent AAV production) were used as negative control samples. Without addition of Shield1 molecule, the AAV production is the same level as the background levels of the qPCR results coming from the negative control (solid red bar on the left vs gray bar at the right). Upon addition of Shield1 molecule the titer levels increased around 3-fold proving the regulation of AAV production using E2A-DBP with degron tag (striped middle bar). FIG. 19B depicts the results of a Western Blot indicating the shift in the protein size of E2A-DBP protein due to the addition of the degron tag. The tagged protein is ˜12 kDa bigger than the untagged DBP protein. The two samples on the left are untagged DBP, while the samples on the right are for DBP with degron.

FIGS. 20A-20B depict that codon-modified Rep with degron motif can be used for AAV production. FIG. 20A shows the modified rep gene under the control of a regulatory element, in particular the TRE3G-Tet system. The construct shown here has large Rep protein produced only by the codon-modified Rep construct. Small Rep is expressed from a separate region on the same plasmid. Small rep is also under TRE3G and degron control, on the same plasmid. The samples from the cells with higher AAV titer are those treated with Shield1 and Dox (FIG. 20B). This shows the inducible properties of multiple degron domains and Tet promoters on multiple AAV genes, including a modified rep gene, within the context of the methods disclosed herein.

### 5. DETAILED DESCRIPTION

The presently disclosed subject matter relates to compositions and methods for the regulation of recombinant rAAV production in cell culture. In particular, the presently disclosed subject matter relates to compositions and methods to overcome AAV Rep protein-mediated cytotoxicity by reversible post-translational regulation of the expression of AAV Rep protein.

In one aspect, the subject matter of the present disclosure is directed to cell culture methods for the post-translational regulation of the expression of an rAAV. In certain embodiments, reversible post-translational regulation of AAV Rep protein expression in the cell culture is achieved by the fusion of a degradation domain to the AAV Rep protein. In certain embodiments, the fusion of a degradation domain to the AAV Rep protein allows for the regulated degradation of AAV Rep protein based on the presence or absence of a degradation ligand in the cell culture. In certain embodiments, the regulated degradation of AAV Rep protein based on the presence or absence of a degradation ligand results in post-translational regulation of the expression of an rAAV in the cell culture.

In another aspect, the subject matter of the present disclosure is directed to rAAV producing cells. For example, but not limitation, the present disclosure is directed to rAAV producing cells wherein the expression of the AAV Rep protein is regulated by the fusion of a degradation domain to the AAV Rep protein.

In another aspect, reversible post-translational regulation of AAV Helper protein expression in the cell culture is achieved by the fusion of a degradation domain to the AAV Helper protein. In certain embodiments, the fusion of a degradation domain to the AAV Helper protein allows for the regulated degradation of AAV Helper protein based on the presence or absence of a degradation ligand in the cell culture. In certain embodiments, the regulated degradation of AAV Helper protein based on the presence or absence of a degradation ligand results in post-translational regulation of the expression of an rAAV in the cell culture.

For clarity, but not by way of limitation, the detailed description of the presently disclosed subject matter is divided into the following subsections:
5.1. Definitions
5.2. Methods of Regulating Expression of AAV Using Degradation Domains
5.3. rAAV Producing Cells

5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one” and “one or more than one.”

The terms “comprise(s),” “include(s),” “having,” “has,” “can,” “contain(s)” and variants thereof, as used herein, are intended to be open-ended transitional phrases, terms or words that do not preclude the possibility of additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

In certain embodiments, the cells of the present disclosure harbor a chromosomally integrated rep gene but require helper virus function in order to express Rep protein. “Helper virus” or “helper virus function” or “AAV Helper” as used herein refers to at least one of adenovirus (Ad) E1 (e.g., Ad E1a or Ad E1b), Ad E2A, Ad E4 and VA RNA, or to corresponding functions of other viruses, such as herpesviruses and poxviruses, which are able to impart helper function to support replication and packaging of AAV vector genomes. In particular embodiments, a hybrid virus made of adenovirus with an E1/E3 deletion, but containing Ad E2A, Ad E4 and VA RNA which provide helper virus function, as well as AAV ITRs flanking a heterologous nucleic acid. In other embodiments, hybrid viruses comprise helper virus functions from herpesvirus or poxvirus, along with AAV ITRs flanking a heterologous nucleic acid.

As used herein, the term “helper virus function(s)” refers to function(s) encoded in a helper virus genome which allow rAAV vector genome replication and packaging (in conjunction with Rep and Cap). As disclosed herein, “helper virus function” may be provided in a number of different ways. For example, helper virus function can be provided by a virus or, for example, provided by polynucleotide sequences encoding the requisite helper function(s) to a cell in trans. In another example, a plasmid or other expression vector comprising polynucleotide sequences encoding one or more viral (e.g., adenoviral) proteins provides helper function when after transfection into a cell line of the invention along with a rAAV vector genome allows rAAV vector genome replication and packaging into rAAV vector particles. In certain embodiments, a helper virus function is provided by a virus selected from adenovirus, herpesvirus, poxvirus, or a hybrid virus thereof. In certain embodiments, a helper virus function comprises one or more viruses, vectors or plasmids that provide the helper virus function. In certain embodiments, the helper virus function comprises at least one of Ad E1 protein (e.g., Ad E1a protein or Ad E1b protein), Ad E2A protein, Ad E4 protein and Ad VA RNA. In certain embodiments the degradation ligand-dependent degradation domain is fused to a Helper protein.

As used herein, the term “AAV protein” refers to any wild-type or modified protein that is derived from the AAV genome and required for AAV production. “AAV protein” includes any form of the Rep, Cap, or Helper proteins, whether wild-type or modified. Modifications of the wild-type AAV genes need not result in a change in amino acid sequence of the expressed protein. As used herein “AAV fusion protein” refers to a fusion protein comprising an AAV protein where the amino acid sequence of the AAV protein is fused, directly or via a linker, to another amino acid sequences, e.g., a degron sequence.

The term “vector” refers to small carrier nucleic acid molecule, e.g., a plasmid, a virus (e.g., AAV vector), or other vehicle, that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell.

A vector nucleic acid sequence generally contains at least an origin of replication for propagation in a cell and optionally additional elements, such as a heterologous polynucleotide sequence, expression control element (e.g., a promoter, enhancer), intron, an inverted terminal repeat (ITR), selectable marker (e.g., antibiotic resistance), polyadenylation signal.

A viral vector is derived from or based upon one or more nucleic acid elements that comprise a viral genome. A particular viral vector is an adeno-associated virus (AAV) vector.

The term “recombinant,” as a modifier of vector, such as recombinant AAV vector, as well as a modifier of sequences such as recombinant polynucleotides and polypeptides, means that the compositions have been manipulated (i.e., engineered) in a fashion that generally does not occur in nature. A particular example of a recombinant AAV vector would be where a click acid sequence that is not normally present in the wild-type AAV genome (e.g., a heterologous nucleic acid sequence) is inserted within the AAV genome. Although the term “recombinant” is not always used herein in reference to AAV vectors, as well as sequences such as polynucleotides, recombinant forms including polynucleotides, are expressly included in spite of any such omission.

A “recombinant AAV vector” or “rAAV” is derived from the wild type (wt or wild-type) genome of AAV by using molecular methods to remove the wild type genome from the AAV genome, and replacing with a non-native nucleic acid sequence, referred to as a heterologous nucleic acid. Typically, for AAV one or both inverted terminal repeat (ITR) sequences of AAV genome are retained in the AAV vector. rAAV is distinguished from an AAV genome, since all or a part of the AAV genome has been replaced with a non-native sequence with respect to the AAV genomic nucleic acid. Incorporation of a non-native sequence therefore defines the AAV vector as a “recombinant” vector, which can be referred to as a “rAAV vector.”

A rAAV sequence can be packaged—referred to herein as a “particle”—for subsequent infection (transduction) of a cell, ex vivo, in vitro or in vivo. Where a recombinant AAV vector sequence is encapsidated or packaged into an AAV particle, the particle can also be referred to as a “rAAV vector” or “rAAV particle.” Such rAAV particles include proteins that encapsidate or package the vector genome. In the case of AAV, they are referred to as capsid proteins.

A vector “genome” refers to the portion of the recombinant plasmid sequence that is ultimately packaged or encapsidated to form a viral (e.g., AAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsidated into virus (e.g., AAV) particles. Thus, a vector “genome” refers to the nucleic acid that is packaged or encapsidated by virus (e.g., AAV).

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides (e.g., variant nucleic acid). The nucleic acids such as cDNA, genomic DNA, RNA, and fragments thereof which may be single- or double-stranded.

Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

A “transgene” is used herein to conveniently refer to a heterologous nucleic acid that is intended or has been introduced into a cell or organism. Transgenes include any heterologous nucleic acid, such as a gene that encodes a polypeptide or protein or encodes an inhibitory RNA.

A heterologous nucleic acid can be introduced/transferred by way of vector, such as AAV, “transduction” or “transfection” into a cell. The term “transduce” and grammatical variations thereof refer to introduction of a molecule such as an rAAV vector into a cell or host organism. The introduced heterologous nucleic acid may also exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated and the introduced protein expressed, or nucleic acid transcribed. For gene therapy uses and methods, a transduced cell can be in a subject.

An “expression control element” is a type of regulatory element and refers to nucleic acid sequence(s) that influence expression of an operably linked nucleic acid. Control elements, including expression control elements as set forth herein such as promoters and enhancers. Vector sequences including AAV vectors can include one or more “expression control elements.” Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron).

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed nucleic acid sequence. A promoter typically increases an amount expressed from operably linked nucleic acid as compared to an amount expressed when no promoter exists.

Another type of regulatory element includes Tet. In Tet dependent induction, expression from a target transgene is dependent on the inducible promoter. Promoter can be regulated by the levels of the tetracycline or tetracycline derivatives such as doxycycline (Dox). The activation of Tet-On promoters depends on the presence of an additional activator protein that can bind to the promoters in the presence of Dox. On the contrary, the transcription is inactive in the presence of Dox for the Tet-Off system. Other examples of inducible systems include Cumate, abscisic acid (ABA), Rapamycin, tamoxifen inducible systems. The degradation ligand-dependent degradation domain disclosed herein can also be used with Cre-LoxP, CRISPR, riboswitch and light-switchable transgene systems as well. An “enhancer” as used herein can refer to a sequence that is located adjacent to the heterologous nucleic acid. Enhancer elements are typically located upstream of a promoter element but also function and can be located downstream of or within a sequence. Enhancer elements typically increase expressed of an operably linked nucleic acid above expression afforded by a promoter element.

Expression control elements herein, such as promoters, are typically positioned at a distance away from the transcribed sequence. In particular embodiments, an expression control element such as a promoter is positioned at least about 25 nucleotides 5′ of the rep gene start codon, is positioned about 25-5,000 nucleotides 5′ of the rep gene start codon, is positioned about 250-2,500 nucleotides 5′ of the rep gene start codon, is positioned about 500-2,000 nucleotides 5′ of the rep gene start codon, is positioned about 1,000-1,900 nucleotides 5′ of the rep gene start codon, is positioned about 1,500-1,900 nucleotides 5′ of the rep gene start codon, is positioned about 1,600-1,800 nucleotides 5′ of the rep gene start codon, is positioned about 1,700-1,800 nucleotides 5′ of the rep gene start codon, or is positioned about 1,750 nucleotides 5′ of the rep gene start codon.

Expression control elements include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements (see, e.g., Boshart et al., Cell, 41:521-530 (1985)), the SV40 promoter, the dihydrofolate reductase promoter, the cytoplasmic β-actin promoter and the phosphoglycerol kinase (PGK) promoter.

Expression control elements also include the native elements(s) for the heterologous polynucleotide. A native control element (e.g., promoter) may be used when it is desired that expression of the heterologous polynucleotide should mimic the native expression. Other native control elements, such as introns, polyadenylation sites or Kozak consensus sequences may also be used.

The term “operably linked” means that the regulatory sequences necessary for expression of a nucleic acid sequence are placed in the appropriate positions relative to the sequence so as to effect expression of the nucleic acid sequence. This same definition is sometimes applied to the arrangement of nucleic acid sequences and transcription control elements (e.g. promoters, enhancers, and termination elements) in an expression vector, e.g., rAAV vector.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

As disclosed herein, a nucleic acid spacer sequence positioned between an expression control element and an AAV rep gene can substantially reduce or eliminate expression of the rep gene thereby in turn reducing or eliminating expression of the Rep protein and allowing cells to survive even while the cells also express adenovirus E1a protein. Addition of helper virus function to such cells, such as provided by a hybrid virus, adenovirus, poxvirus or herpesvirus, can overcome the attenuating effect of the spacer nucleic acid on rep gene expression and in turn drive expression of rep gene thereby providing Rep protein expression.

Additional elements for rAAV vectors include, without limitation, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. For a nucleic acid sequence less than 4.7 kb, the filler or stuffer polynucleotide sequence has a length that when combined (e.g., inserted into a vector) with the sequence has a total length between about 3.0-5.5 kb, or between about 4.0-5.0 kb, or between about 4.3-4.8 kb.

Where a wild type heterologous nucleic acid or transgene is too large to be packaged within an AAV vector particle, the heterologous nucleic acid may be provided in modified, fragmented or truncated form for packaging in and delivery by an AAV vector, such that a functional protein or nucleic acid product, such as a therapeutic protein or nucleic acid product, is ultimately provided.

In some embodiments, the heterologous nucleic acid that encodes a protein (e.g., therapeutic protein) is provided in modified or truncated forms or the heterologous nucleic acid is provided in multiple constructs, delivered by separate and multiple AAV vectors.

In certain aspects, the heterologous nucleic acid is provided as a truncated variant that maintains functionality of the encoded protein (e.g., therapeutic protein), including removal of portions unnecessary for function, such that the encoding heterologous polynucleotide is reduced in size for packaging in an AAV vector.

In certain aspects the heterologous nucleic acid is provided in split AAV vectors, each providing nucleic acid encoding different portions of a protein (e.g., therapeutic protein), thus delivering multiple portions of a protein (e.g., therapeutic protein) which assemble and function in the cell.

In other aspects, the heterologous nucleic acid is provided by dual AAV vectors using overlapping, trans-splicing or hybrid trans-splicing dual vector technology. In certain embodiments, two overlapping AAV vectors are used which combine in the cell to generate a full expression cassette, from which a full-length protein (e.g., therapeutic protein) is expressed.

A “hemostasis related disorder” refers to bleeding disorders such as hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, combined FV/FVIII deficiency, thalassemia, vitamin K epoxide reductase C1 deficiency, or gamma-carboxylase deficiency; bleeding associated with trauma, injury, thrombosis, thrombocytopenia, stroke, coagulopathy, or disseminated intravascular coagulation (DIC); over-anticoagulation associated with heparin, low molecular weight heparin, pentasaccharide, warfarin, or small molecule antithrombotics (i.e., FXa inhibitors); and platelet disorders such as, Bernard Soulier syndrome, Glanzmann thrombasthenia, and storage pool deficiency.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

The term “isolated” does not exclude combinations produced by the hand of man, for example, a rAAV sequence, or rAAV particle that packages or encapsidates an AAV vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or at least 85% by weight, or about 90-99% by weight, of the compound of interest. Purity is measured by methods appropriate for the compound of interest (e.g., chromatographic methods, agarose or polyacrylamide gel electrophoresis, HPLC analysis, and the like).

The phrase “consisting essentially of” when referring to a particular nucleotide sequence or amino acid sequence means a sequence having the properties of a given SEQ ID NO. For example, when used in reference to an amino acid sequence, the phrase includes the sequence per se and molecular modifications that would not affect the basic and novel characteristics of the sequence.

The term “identity,” “homology” and grammatical variations thereof, mean that two or more referenced entities are the same, when they are “aligned” sequences. Thus, by way of example, when two protein sequences are identical, they have the same amino acid sequence, at least within the referenced region or portion. Where two nucleic acid sequences are identical, they have the same nucleic acid sequence, at least within the referenced region or portion. The identity can be over a defined area (region or domain) of the sequence.

An “area” or “region” of identity refers to a portion of two or more referenced entities that are the same. Thus, where two protein or nucleic acid sequences are identical over one or more sequence areas or regions they share identity within that region. An “aligned” sequence refers to multiple protein (amino acid) or nucleic acid sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous amino acids or nucleic acids, e.g., 6, 7, 8, 9, 10, 11, 12, 13, 14, 15, 16, 17, 18, 19, 20, etc. contiguous nucleic acids or amino acids. In additional embodiments, the length of the sequence sharing identity is 21 or more contiguous amino acids or nucleic acids, e.g., 21, 22, 23, 24, 25, 26, 27, 28, 29, 30, 31, 32, 33, 34, 35, 36, 37, 38, 39, 40, etc. contiguous amino acids or nucleic acids. In further embodiments, the length of the sequence sharing identity is 41 or more contiguous amino acids or nucleic acids, e.g., 42, 43, 44, 45, 45, 47, 48, 49, 50, etc., contiguous amino acids or nucleic acids. In yet further embodiments, the length of the sequence sharing identity is 50 or more contiguous amino acids or nucleic acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous amino acids or nucleic acids.

The extent of identity (homology) or “percent identity” between two sequences can be ascertained using a computer program and/or mathematical algorithm. For purposes of this invention comparisons of nucleic acid sequences are performed using the GCG Wisconsin Package version 9.1, available from the Genetics Computer Group in Madison, Wisconsin. For convenience, the default parameters (gap creation penalty=12, gap extension penalty=4) specified by that program are intended for use herein to compare sequence identity. Alternately, the Blastn 2.0 program provided by the National Center for Biotechnology Information (found on the world wide web at ncbi.nlm.nih.gov/blast/; Altschul et al., 1990, J Mol Biol 215:403-410) using a gapped alignment with default parameters, may be used to determine the level of identity and similarity between nucleic acid sequences and amino acid sequences. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Nucleic acid molecules, expression vectors (e.g., AAV vector genomes), plasmids, including nucleic acid encoding modified/variant AAV capsids of the invention and heterologous nucleic acids may be prepared by using recombinant DNA technology methods. The availability of nucleotide sequence information enables preparation of isolated nucleic acid molecules of the invention by a variety of means. For example, nucleic acid sequences can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

5.2. Methods of Regulating AAV Production Using Degradation Domains

In one aspect, the subject matter of the present disclosure is directed to cell culture methods for the post-translational regulation of the expression of an rAAV. In certain embodiments, reversible post-translational regulation of AAV Rep protein expression in the cell culture is achieved by the fusion of a degradation domain (“degron”) to the AAV Rep protein. In certain embodiments, the fusion of a degradation domain to the AAV Rep protein allows for the regulated degradation of AAV Rep protein based on the presence or absence of a degradation ligand or other “inhibitor”, e.g., a small molecule ligand that binds to the degron to modify its rate of degradation or an inhibitor such as light or temperature, that modifies the rate of degradation of the degron. In certain embodiments, the regulated degradation of AAV Rep protein based on the presence or absence of a degradation ligand or other inhibitor results in post-translational regulation of the expression of an rAAV in the cell culture. As used herein, the term “degradation ligand-dependent degradation domain” refers to a degron domain that binds a degradation ligand. As used herein, the term “degradation ligand” refers to a ligand that binds the degradation domain. Any suitable degron can be used in connection with the methods of the present disclosure. For example, but not limitation, the degron is modified from a human gene encoding a protein referred to as FK506-binding protein 12 (“FKBP”). In certain embodiments, the degron is derived from FKBP. In certain embodiments, the FKBP variant protein from which the FKBP degron is derived comprises an F36V amino acid substitution. In certain embodiments, the FKBP variant protein from which the FKBP degron is derived comprises an L106P amino acid substitution. In certain embodiments, the FKBP variant protein from which the FKBP degron is derived comprises both an F36V and an L106P amino acid substitution. In certain embodiments, the modifications of FKBP: a) deepen the binding pocket to improve its specificity to a degradation ligand, e.g., Shield1, over FK506; and/or b) make the protein more unstable in the absence of its degradation ligand. dTAG-13 is a small molecule that can target mutant FKBP12 (F36V) sequences for ubiquitin mediated degradation. It can function by linking the targeted protein sequence to a E3 ubiquitin ligase, cereblon. dTAG-13 can lead to degradation of FKBP12-F36V fusion proteins and proteins fused to it. As used herein, the FKBP12 domain is referred to as “FKBP”.

In certain embodiments the degron is a dihydrofolate reductase (DHFR) based degron; an auxin-induced degron (AID) domain; an ornithine decarboxylase (ODC) based degron; a split ubiquitin based degron system; a protease based degron system; a Proteolysis-Targeting Chimeric Molecules (PROTACs) based degron system; an antibody dependent protein degron system; a photosensitive degron (psd); a phosphorylation-dependent degron; or a temperature dependent degron.

In certain embodiments, the degron is regulated by the presence or absence of a degradation ligand or inhibitor. In certain embodiments the degradation ligand is a small molecule ligand. In certain embodiments, e.g., when the degron is a FKBP variant protein, the small molecule ligand is Shield1.

In certain embodiments, e.g., when the degron is a dihydrofolate reductase (DHFR) based degron, the ligand is Trimethoprim (TMP). In certain embodiments, e.g., when the degron is an auxin-induced degron (AID) domain, the ligand is auxin. In certain embodiments, e.g., when the degron is an ornithine decarboxylase (ODC) based degron, the ligand is antizyme. In certain embodiments, e.g., when the degron is a split ubiquitin based degron system, the ligand is rapamycin. In certain embodiments, e.g., when the degron is a protease based degron system, the inhibitor can be an HCV protease inhibitor or TEV protease expression. In certain embodiments, e.g., when the degron is a Proteolysis-Targeting Chimeric Molecules (PROTACs) based degron system, the inhibitor is PROTAC expression. In certain embodiments, e.g., when the degron is an antibody dependent protein degron system, the ligand is a corresponding antibody. In certain embodiments, e.g., when the degron is a photosensitive degron (psd), the inhibitor is light. In certain embodiments, e.g., when the degron is a phosphorylation-dependent degron, the inhibitor is a corresponding kinase activator. In certain embodiments, e.g., when the degron is a temperature dependent degron, the inhibitor is a temperature change.

In certain embodiments, the degron sequence is linked to the C-terminus of an AAV Rep protein. In certain embodiments, the degron sequence is linked to the N-terminus of an AAV Rep protein. In certain embodiments, the degron and the AAV Rep protein are linked through a flexible linker. In certain embodiments, the degron and the AAV Rep protein are linked through a rigid linker. In certain non-limiting embodiments, the flexible linker has the amino acid sequence: GGGGSGGGGSGGGGS. In certain non-limiting embodiments, the rigid linker has the amino acid sequence:

AEAAAKEAAAKEAAAKEAAAKALEAEAAAKEAAAKEAAAKEA AAKA.

In certain embodiments, the methods of the present disclosure are directed to regulating the production of a recombinant adeno-associated (rAAV) virus, where the method comprises: introducing into a mammalian cell an rAAV comprising a gene of interest and a nucleic acid encoding a fusion protein, wherein the fusion protein comprises the Rep protein, a linker, and a degradation ligand-dependent degradation domain, culturing the cell under conditions suitable for producing the rAAV virus; and contacting the cell with a degradation ligand, wherein the degradation ligand binds to the degradation domain to regulate the expression of the Rep protein and thereby regulate the production of rAAV.

In certain embodiments, the cell is a E1a expressing cell. In certain embodiments, the cell is a human cell. In certain embodiments, the cell is a HEK293 cell. In certain embodiments, the cell is a HEK293F cell. In certain embodiments, the cell is a PERC6 cell.

In certain embodiments, the Rep protein is a Rep78, Rep68, Rep52, or Rep40 protein.

The methods of the present disclosure comprise the use of suitable regulatory elements, including promoters to drive the expression of Rep and Cap proteins. In certain embodiments, suitable promoters may be eukaryotic, prokaryotic, or viral promoters. Suitable promoters include non-inducible promoters and non-tissue specific promoters. In certain embodiments, the promoter is an AAV p5 promoter, which in its native state drives Rep protein expression from the rep gene. In certain embodiments, the promoter is the cytomegalovirus (CMV) immediate early promoter/enhancer. Additional nonlimiting examples of suitable promoters include ubiquitous or promiscuous promoters which are capable of driving expression of a polynucleotide in many different cell types. Such elements include, but are not limited to the Rous sarcoma virus (RSV) promoter sequences and the other viral promoters active in a variety of mammalian cell types, or synthetic elements (see, e.g., Boshart el al., Cell, 41:521-530 (1985)), the SV40 promoter, the dihydrofolate reductase promoter, the cytoplasmic b-actin promoter and the phosphoglycerol kinase (PGK) promoter. In certain embodiments, the promoter is selected from the human elongation factor-1αEF1 alpha promoter, the CAG promoter, the CBA promoter, the SFFV promoter, the p19 promoter, and the herpes simplex virus thymidine kinase (HSV-TK) promoter. In certain embodiments promoters may be located proximal or distal to the Rep and Cap genes. In certain embodiments regulatory elements, including promoters, may be in cis or trans to the Rep and Cap genes. In certain embodiments, the methods of the present disclosure are directed to expression of a nucleic acid encoding an AAV Rep protein, wherein the AAV Rep protein is an AAV1 Rep protein, an AAV2 Rep protein, an AAV3 Rep protein, anAAV4 Rep protein, an AAV5 Rep protein, an AAV6 Rep protein, an AAV7 Rep protein, an AAV8 Rep protein, an AAV9 Rep protein, an AAV 10 Rep protein, or an AAV 11 Rep protein. Because the methods of the present disclosure are applicable to any cell type that produces AAV, the AAV can be, e.g., human, avian, bovine, canine, equine, primate, non-primate, ovine, or any derivation thereof.

In certain embodiments, the rAAV produced by the methods of the present disclosure include any viral strain or serotype. In certain, non-limiting, embodiments, an rAAV can be based upon any AAV genome, including, but not limited to AAV1, AAV2, AAV3, AAV4, AAV5, AAV6, AAV7, AAV8, AAV9, AAV9.47, AAV9(hul4), AAV10, AAV11, AAV12, Rh8, Rh10, Rh74, AAV3B, AAV-2i8, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9 variants including AAV9.47 among others), U.S. Pat. No. 7,906,111 (describing AAV9(hul4) among others), U.S. Pat. No. 10,532,111 (describing NP59 among others), U.S. Patent No. U.S. Ser. No. 10/738,087 (describing Anc-80 among others), WO2012/145601, WO2013/158879, WO2015/013313, WO2018/156654, US2013/0059732, U.S. Pat. Nos. 9,169,299 (describing LK03), 9,840,719 (describing RHM4-1), 7,749,492, 7,588,772 (describing DJ and DJ8), and 9,587,282, all of which are incorporated herein by reference in their entireties. rAAV vectors therefore include gene/protein sequences identical to gene/protein sequences characteristic for a particular serotype, as well as mixed serotypes.

In certain embodiments, the nucleic acid encoding AAV Rep and Cap proteins is arranged as in the native AAV genome. In certain embodiments, the nucleic acid encoding AAV Rep and Cap proteins is arranged as in the native AAV genome, except that the nucleic acid encodes a linker and/or a degron sequence at the N-terminus or C-terminus of the AAV Rep protein. In certain embodiments, the nucleic acids encoding AAV Rep and Cap proteins are not arranged, directly or indirectly, in a tandem configuration. In certain embodiments, the nucleic acids encoding AAV Rep and Cap proteins are not covalently linked. Additional suitable arrangements of the AAV Rep and Cap coding sequences can be employed, e.g., the order of the AAV Rep and Cap coding sequences can be reversed relative to their native AAV genomic order, one or both of the AAV Rep and Cap coding sequences can be preceded or followed by a sequence comprising an IRES, a self-cleaving protein (e.g., a 2A peptide) coding sequence, or stuffer region having no function. In certain embodiments, one or both of the AAV Rep and Cap coding sequences can be incorporated into a cell genome. In certain embodiments, one or both of the AAV Rep and Cap coding sequences can be combined with factors that increase DNA sequences that increase episomal plasmid maintenance.

In certain embodiments, a helper virus function is provided by a virus selected from adenovirus, herpesvirus, poxvirus, or a hybrid virus thereof. In certain embodiments, a helper virus function comprises one or more viruses, vectors or plasmids that provide the helper virus function. In certain embodiments, the helper virus function comprises at least one of adenovirus (Ad) E1 protein (e.g., Ad E1a or Ad E1b protein), Ad E2A protein, Ad E4 protein and Ad VA RNA.

The helper virus function can be provided in a number of different ways. In certain embodiments, the helper virus function can be provided by a virus or, for example, provided by polynucleotide sequences encoding the requisite helper function(s) to a cell in trans.

In certain embodiments, the degron sequence is linked to the C-terminus of an adenovirus (Ad) E1 protein (e.g., Ad E1a or Ad E1b protein), Ad E2A protein, or Ad E4 protein. In certain embodiments, the degron sequence is linked to the N-terminus of an Ad E1 protein (e.g., Ad E1a or Ad E1b protein), Ad E2A protein, or Ad E4 protein. In certain embodiments, the degron and the Ad E1 protein (e.g., Ad E1a or Ad E1b protein), Ad E2A protein, or Ad E4 protein are linked through a flexible linker. In certain embodiments, the degron and the Ad E1 protein (e.g., Ad E1a or Ad E1b protein), Ad E2A protein, or Ad E4 protein are linked through a rigid linker.

In certain embodiments, two or more AAV proteins, e.g., Rep, Cap, and/or Helper proteins, are independently linked to a degron sequence to generate two or more AAV protein-degron fusion proteins. In certain embodiments, each of the two or more AAV protein-degron fusion proteins comprise a degron at the AAV protein's C-terminus or N-terminus. In certain embodiments, each of the two or more AAV protein-degron fusion proteins comprise a different degron sequence. In certain embodiments, each the two or more AAV protein-degron fusion proteins comprise the same degron sequence. In certain embodiments, the linkage between the AAV protein and its respective degron is a flexible linker. In certain embodiments, the linkage between the AAV protein and its respective degron is a rigid linker.

In certain embodiments, the two or more AAV protein-fusion proteins can be encoded by sequences on the same vector, e.g., plasmid. In certain embodiments, the two or more AAV protein-degron fusion proteins can be encoded by sequences on separate vectors, e.g., a first plasmid comprises the coding sequence for the first AAV protein-degron fusion and a second plasmid comprises the coding sequence for the second AAV protein-degron fusion. In certain embodiments, the expression of at least one of the AAV protein-degron fusion proteins is under the control of a regulatory element. In certain embodiments, the expression of at least two of the AAV protein-degron fusion proteins is under the control of a regulatory element. In certain embodiments, the expression of multiple AAV protein-degron fusion proteins are each under the control of a different regulatory element. In certain embodiments, the expression of all of the AAV protein-degron fusion proteins is under the control of a regulatory element. In certain embodiments, the regulatory element is a promoter. In certain embodiments the regulatory element is a Tet response element

In certain embodiments, the cell of the present disclosure comprises acid sequence that is not normally present in the wild-type AAV genome, e.g., a heterologous nucleic acid sequence, also referred to herein as a gene of interest or (GOI). In certain non-limiting embodiments, the GOI comprises a nucleic acid sequence encoding a therapeutic protein or an inhibitory nucleic acid sequence. In certain embodiments, the GOI can be introduced/transferred by way of vector, such as AAV transduction or transfection into a cell. In certain embodiments, the introduced GOI can also exist in the recipient cell or host organism extrachromosomally, or only transiently.

In certain embodiments, the GOI encodes a protein (e.g., therapeutic protein) that is provided in modified or truncated forms or the GOI is provided in multiple constructs, delivered by separate and multiple AAV vectors.

In certain embodiments, the GOI is provided as a truncated variant that maintains functionality of the encoded protein (e.g., therapeutic protein), including removal of portions unnecessary for function, such that the GOI is reduced in size for packaging in an AAV vector.

In certain embodiments, the GOI is provided in split AAV vectors, each providing nucleic acid encoding different portions of a protein (e.g., therapeutic protein), thus delivering multiple portions of a protein (e.g., therapeutic protein) which assemble and function in the cell.

In certain embodiments, the GOI is provided by dual AAV vectors using overlapping, trans-splicing or hybrid trans-splicing dual vector technology. In certain embodiments, two overlapping AAV vectors are used which combine in the cell to generate a full expression cassette, from which a full-length protein (e.g., therapeutic protein) is expressed.

5.3. AAV Producing Cells

In another aspect, the subject matter of the present disclosure is directed to rAAV producing cells. For example, but not limitation, the present disclosure is directed to rAAV producing cells wherein the expression of the AAV Rep protein is regulated by the fusion of a degradation domain to the AAV Rep protein.

The embodiments of the rAAV producing cells of the present disclosure include any cell type or system that can produce rAAV or AAV. Several examples of various systems have been previously described, e.g., Conlon and Mavilio, Mol. Therapy, 8:181-182 (2018), which is incorporated herein by reference in its entirety. Embodiments of the present disclosure include making rAAV through transient transfection of plasmids in mammalian cells, production of rAAV in stable cell lines, rAAV through herpes simplex virus in mammalian cells, and rAAV production through baculovirus in Sf9 cells.

In certain embodiments, the rAAV producing cells of the present disclosure are eukaryotic cells. In certain embodiments, the rAAV producing cells of the present disclosure are animal cells. In certain embodiments, the rAAV producing cells of the present disclosure are insect cells. In certain embodiments, the rAAV producing cells of the present disclosure mammalian cells. In certain embodiments, the rAAV producing cells of the present disclosure are human cells. In certain embodiments, the rAAV producing cells of the present disclosure human embryonic kidney (HEK) cells. In certain embodiments, the rAAV producing cells of the present disclosure are HEK293 cells, HEK293F cells, or Expi293 cells.

In certain embodiments, the rAAV producing cells of the present disclosure are Chinese hamster ovary (CHO) cells.

In certain embodiments, the rAAV producing cells of the present disclosure are insect (Sf9) cells.

In certain embodiments, the rAAV producing cells of the present disclosure do not express SV40 large T antigen.

In certain embodiments, the rAAV producing cells of the present disclosure are suspension cells. In certain embodiments, the rAAV producing cells of the present disclosure are adherent cells.

In certain embodiments, the rAAV producing cells of the present disclosure can be cultured at a cell density of at least about 1×10⁶, at least about 5×10⁶, at least about 1×10⁷ or at least about 2×10⁷ cells/mL. In certain embodiments, the rAAV producing cells of the present disclosure can be cultured at a cell density from about 1×10⁶−5×10⁶, from about 5×10⁶−1×10⁷, or from about 1×10⁷−2×10⁷ cells/mL.

In certain embodiments, the rAAV producing cells of the present disclosure are present in a culture or growth medium.

In certain embodiments, the rAAV producing cells of the present disclosure are in medium suitable for storage. In certain embodiments, the rAAV producing cells of the present disclosure are in a medium suitable for long-term storage at or below 0°, at or below −30°, at or below −80° or at or below −160° C.

In certain embodiments, the present disclosure is directed to a mammalian rAAV producing cell, wherein the cell comprises a nucleic acid encoding a fusion protein comprising the Rep protein, a linker, and a degradation ligand-dependent degradation domain. In certain embodiments, the degradation ligand-dependent degradation domain is a FKBP variant protein. In certain embodiments, the degradation ligand is a small molecule ligand. In certain embodiments, the small molecule is Shield1.

In certain embodiments, the mammalian rAAV producing cell of the present disclosure is an E1a-expressing cell. In certain embodiments, the E1a-expressing cell is a HEK293 cell.

In certain embodiments, the ligand-dependent degradation domain of the mammalian rAAV producing cell of the present disclosure is fused via a linker to the C-terminal end of the Rep protein. In certain embodiments, the ligand-dependent degradation domain of the mammalian rAAV producing cell of the present disclosure is fused via a linker to the N-terminal end of the Rep protein. In certain embodiments, the linker is a flexible linker. In certain non-limiting embodiments, the flexible linker has the amino acid sequence: GGGGSGGGGSGGGGS. In certain embodiments, the linker is a rigid linker. In certain non-limiting embodiments, the rigid linker has the amino acid sequence:

AEAAAKEAAAKEAAAKEAAAKALEAEAAAKEAAAK EAAAKEAAAKA.

In certain embodiments, the mammalian rAAV producing cell of the present disclosure comprises a nucleic acid encoding AAV Rep and Cap proteins arranged as in the native AAV genome. In certain embodiments, the nucleic acid encoding AAV Rep and Cap proteins is arranged as in the native AAV genome, except that the nucleic acid encodes a linker and/or a degron sequence at the N-terminus or C-terminus of the AAV Rep protein. In certain embodiments, the nucleic acids encoding AAV Rep and Cap proteins are not arranged, directly or indirectly, in a tandem configuration. In certain embodiments, the nucleic acids encoding AAV Rep and Cap proteins are not covalently linked. Additional suitable arrangements of the AAV Rep and Cap coding sequences can be employed, e.g., the order of the AAV Rep and Cap coding sequences can be reversed relative to their native AAV genomic order, one or both of the AAV Rep and Cap coding sequences can be preceded or followed by a sequence comprising an IRES, a self-cleaving protein (e.g., a 2A peptide) coding sequence, or stuffer region having no function. In certain embodiments, one or both of the AAV Rep and Cap coding sequences can be incorporated into a cell genome. In certain embodiments, one or both of the AAV Rep and Cap coding sequences can be combined with factors that increase DNA sequences that increase episomal plasmid maintenance.

In certain embodiments, the mammalian rAAV producing cell of the present disclosure comprise a virus capable of helper virus function. In certain embodiments, the virus is selected from adenovirus, herpesvirus, poxvirus, or a hybrid virus thereof. In certain embodiments, the mammalian rAAV producing cell of the present disclosure comprises one or more viruses, vectors or plasmids that provide the helper virus function. In certain embodiments, the helper virus function comprises at least one of adenovirus (Ad) E1 protein (e.g., Ad E1a or Ad E1b protein), Ad E2A protein, Ad E4 protein and Ad VA RNA.

In certain embodiments, the mammalian rAAV producing cell of the present disclosure comprises a GOI. In certain non-limiting embodiments, the GOI comprises a nucleic acid sequence encoding a therapeutic protein or an inhibitory nucleic acid sequence. In certain embodiments, the GOI can be introduced/transferred by way of vector, such as AAV transduction or transfection into a cell. In certain embodiments, the introduced GOI can also exist in the recipient cell or host organism extrachromosomally, or only transiently.

In certain embodiments, the GOI encodes a protein (e.g., therapeutic protein) that is provided in modified or truncated forms or the GOI is provided in multiple constructs, delivered by separate and multiple AAV vectors.

In certain embodiments, the GOI is provided as a truncated variant that maintains functionality of the encoded protein (e.g., therapeutic protein), including removal of portions unnecessary for function, such that the GOI is reduced in size for packaging in an AAV vector.

In certain embodiments, the GOI is provided in split AAV vectors, each providing nucleic acid encoding different portions of a protein (e.g., therapeutic protein), thus delivering multiple portions of a protein (e.g., therapeutic protein) which assemble and function in the mammalian rAAV producing cell of the present disclosure.

In certain embodiments, the GOI is provided by dual AAV vectors using overlapping, trans-splicing or hybrid trans-splicing dual vector technology. In certain embodiments, two overlapping AAV vectors are used which combine in the mammalian rAAV producing cell of the present disclosure to generate a full expression cassette, from which a full-length protein (e.g., therapeutic protein) is expressed.

Non-limiting examples of heterologous nucleic acids encoding gene products (e.g., therapeutic proteins) which are useful in accordance with the invention include those that may be used in the treatment of a disease or disorder including, but not limited to, “hemostasis” or blood clotting disorders such as hemophilia A, hemophilia A patients with inhibitory antibodies, hemophilia B, deficiencies in coagulation Factors, VII, VIII, IX and X, XI, V, XII, II, von Willebrand factor, combined FV/FVIII deficiency, thalassemia, vitamin K epoxide reductase CI deficiency, gamma-carboxylase deficiency; anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e. FXa inhibitors); and platelet disorders such as, Bernard Soulier syndrome, Glanzman thromblastemia, and storage pool deficiency.

In certain embodiments, the disease or disorder affects or originates in the central nervous system (CNS). In certain embodiments, the disease is a neurodegenerative disease. In certain embodiments, the CNS or neurodegenerative disease is Alzheimer's disease, Huntington's disease, ALS, hereditary spastic hemiplegia, primary lateral sclerosis, spinal muscular atrophy, Kennedy's disease, a polyglutamine repeat disease, or Parkinson's disease. In certain embodiments, the CNS or neurodegenerative disease is a polyglutamine repeat disease. In certain embodiments, the polyglutamine repeat disease is a spinocerebellar ataxia (SCA1, SCA2, SCA3, SCA6, SCA7, or SCA17).

In certain embodiments, the AAV particles comprise a heterologous nucleic acid encoding a gene product selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor a (TGFa), platelet-derived growth factor (PDGF), insulin growth factors I and II (IGF-I and IGF-II), TGFp, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

In certain embodiments, the AAV particles comprise a heterologous nucleic acid encoding a gene product selected from the group consisting of thrombopoietin (TPO), interleukins (IL1 through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

In certain embodiments, the AAV particles comprise a heterologous nucleic acid encoding a gene product selected from the group consisting of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor V, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, RPE65, H-protein, T-protein, a cystic fibrosis transmembrane regulator (CFTR) sequence, and a dystrophin cDNA sequence

In certain embodiments, the AAV particles comprise a heterologous nucleic acid encoding a polypeptide, a nucleic acid that encodes a protein or is transcribed into a transcript of interest, or nucleic acid, selected from the group consisting of a siRNA, an antisense molecule, miRNA a ribozyme and a shRNA.

In certain embodiments, the AAV particles comprise a heterologous nucleic that encodes a protein selected from the group consisting of GAA (acid alpha-glucosidase) for treatment of Pompe disease; ATP7B (copper transporting ATPase2) for treatment of Wilson's disease; alpha galactosidase for treatment of Fabry's disease; ASS1 (arginosuccinate synthase) for treatment of citrullinemia Type 1; beta-glucocerebrosidase for treatment of Gaucher disease Type 1; beta-hexosaminidase A for treatment of Tay Sachs disease; SERPING1 (C1 protease inhibitor or C1 esterase inhibitor) for treatment of hereditary angioedema (HAE), also known as C1 inhibitor deficiency type I and type II); and glucose-6-phosphatase for treatment of glycogen storage disease type I (GSDI).

In certain embodiments, a heterologous nucleic acid encodes CFTR (cystic fibrosis transmembrane regulator protein), a blood coagulation (clotting) factor (Factor XIII, Factor IX, Factor VIII, Factor X, Factor VII, Factor VIIa, protein C, etc.) a gain of function blood coagulation factor, an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor −3 and −4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), Rab escort protein 1 (choroideremia), LCA 5 (LCA-Lebercilin), ornithine ketoacid aminotransferase (gyrate atrophy), retinoschisin 1 (X-linked retinoschisis), USH1C (Usher's Syndrome 1C), X-linked retinitis pigmentosa GTPase (XLRP), MERTK (AR forms of RP: retinitis pigmentosa), DFNB1 (connexin 26 deafness), ACHM 2, 3 and 4 (achromatopsia), PKD-1 or PKD-2 (polycystic kidney disease), TPP1, CLN2, a sulfatase, N-acetylglucosamine-1-phosphate transferase, cathepsin A, GM2-AP, NPC1, VPC2, a sphingolipid activator protein, one or more zinc finger nucleases for genome editing, or one or more donor sequences used as repair templates for genome editing.

Nucleic acid molecules, vectors such as cloning, expression vectors (e.g., vector genomes) and plasmids, may be prepared using recombinant DNA technology methods. The availability of nucleotide sequence information enables preparation of nucleic acid molecules by a variety of means. For example, a heterologous nucleic acid encoding Factor IX (FIX) comprising a vector or plasmid can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

### EXAMPLES

The following examples are merely illustrative of the presently disclosed subject matter and should not be considered as limitations in any way.

### Example 1: Rep-Degron Constructs and rAAV Production

Various constructs were designed for the development of mechanism for controllable Rep accumulation and higher rAAV production. Rep transgene was tagged at N-terminus or C-terminus with a destabilization domain (degron) to confer instability and target the translated Rep protein product to proteasome for degradation. The Rep protein degradation can be reversed by addition of the Shield1 ligand. The degron was modified from a human gene encoding FK506-binding protein 12 (“FKBP12”). It was previously shown that F36V modifications of the protein improves its specificity to Shield1 over FK506 (Clackson et al, PNAS 1998). Subsequent efforts to create a more unstable degron motif resulted in L107P mutation (Banaszynski et al, Cell 2006). The amino acid sequence of the degron that was used in the present example, referred to herein as “FKBP”, having a F36V and an L107P mutation, was:

MGVQVETISPGDGRTFPKRGQTCVVHYTGMLEDGKK
VDSSRDRNKPFKFMLGKQEVIRGWEEGVAQMSVGQ
RAKLTISPDYAYGATGHPGIIPPHATLVFDVELLKPE

Production of rAAV: Recombinant AAV were produced by transfection of human embryonic kidney (HEK293) cells grown in 6-well plates. The cells at 70-90% confluency were transfected with 3 μg of total DNA using jetOPTIMUS® DNA (PolyPlus)transfection reagent. The positive control rAAV produced with LK03 Rep/Cap plasmid were produced via triple transfection method. The plasmids were added in 1:1:1 ratio (1 helper: 1 ITR plasmid: 1 rep/cap plasmids). The rAAV plasmids produced with Split Rep/Cap plasmids were produced with quadruple plasmid transfection method where Rep and Cap plasmids were used in equal amounts to each other, but half of the helper and ITR plasmids (1 helper: 1 ITR plasmid: 0.5 Rep: 0.5 cap) unless stated otherwise. For the degron containing constructs, wt Rep or Rep/Cap plasmids were replaced with the degron containing Rep or Rep/Cap plasmids. Following transfection, cell culture media was replaced the next day. Small molecule Shield-1 (Takara) was added to the media of the corresponding samples after media change and cells were grown an additional 24-36 hours to allow for AAV production. The AAV in the crude cell lysate extract or in the media of the HEK293 cells were used to transduce the target cells.

For the use of rAAV in the crude extract, the cells were resuspended in the cell culture medium they were growing in and the suspension was transferred to 1.5 ml tubes. In lieu of detergent-based lyses, cells were lysed by four consecutive freeze and thaw cycles moving between dry ice and a 37° C. water bath. Samples were vortexed following each thaw cycle to enhance cell lysis and promoter viral particle release. Upon completion of lysis steps, the samples were spin down at 13,500×g for 10 minutes in an Eppendorf centrifuge set to 4° C. The supernatant containing rAAV particles was transferred to a fresh tube for use in transduction assays.

Transduction of cells: Huh7 cells were transduced with equivalent volume of cell lysate or the cell media containing the AAV vectors. Briefly, Huh7 cells were seeded into 24-well plates one day prior to transduction. Up to 50 ul of the unpurified viral preparations from HEK293 were added into culture medium of Huh7 cells at. Transduction efficiency was assessed in biological duplicates or triplicates. Culture media was replaced the following day, and samples were analyzed for transgene expression 48-72 hours after transduction. For detection of Luciferase expression, Huh7 cells were lysed in passive lysis buffer (Promega) and each biological sample was divided and plated to four wells of 96-well luminescence assay plate. Renilla Luciferase levels were determined using the Renilla Luciferase Assay Kit (Promega) and analyzed in a microplate luminometer (Spectramax) fitted with injectors.

Cloning of the plasmid constructs: The cloning of constructs having degron motifs were done using Gibson Assembly method. Briefly, the designed degron-linker sequences with desired homology regions were ordered as gBlocks fragments from Integrated DNA Technologies. The backbone containing Rep only plasmids or Rep/Cap plasmids and gblocks were joined using NEBuilder Hifi DNA Assembly Kit (New England Biolabs, #E5520). Upon plasmid purification and sequence verification, the plasmids with correct sequences were used for the rAAV production experiment.

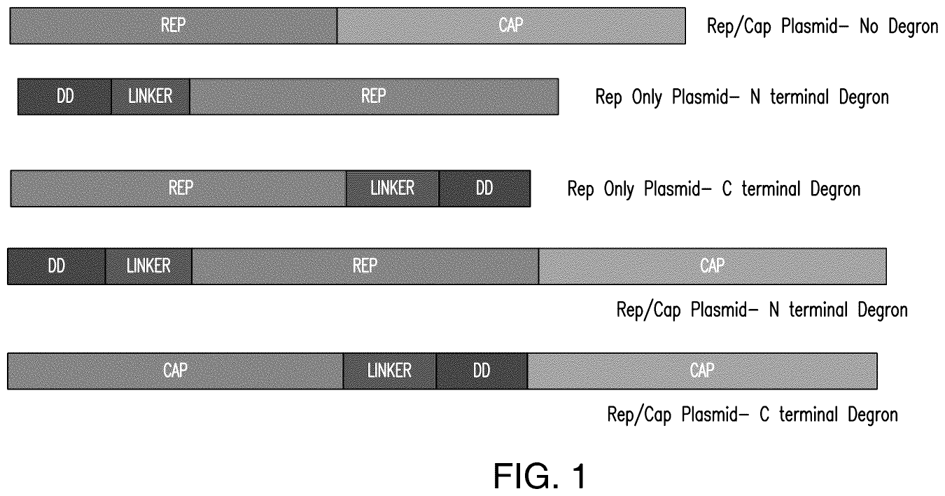

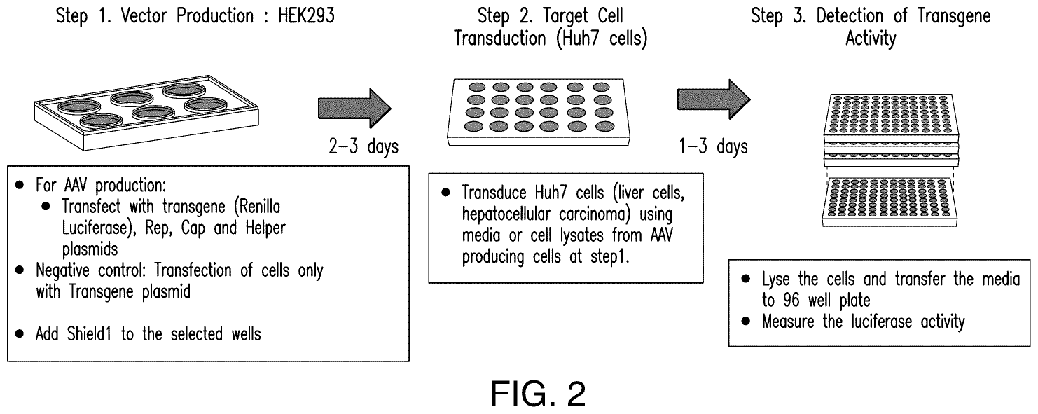

As shown in FIGS. 1 and 2, the REP and Cap genes were cloned either into the same or into separate plasmids. The aim was to regulate Rep expression without altering Cap expression. Plasmids were developed having the degron sequences cloned to the C-terminal end or to the N-terminal end of the Rep sequence (FIG. 1). Two types of linkers were tested; rigid and flexible linkers. The P40 promoter was used to drive expression of Cap. For Rep, the p5 promoter was used in certain instances while the CMV promoter was used in others, as indicated.

Transfection of cells with Transgene plasmid only, as shown in FIGS. 1 and 2, was used as a negative control. Shield1 was added to selected wells (FIG. 2). Huh7 cells (liver cells, hepatocellular carcinoma) were transduced using media or cell lysates from the rAAV producing HEK293 cells. The Huh7 cells were then lysed and luciferase activity was measured for the detection of transgene activity.

### Example 2: rAAV Produced by Transfection of Rep and Cap on Separate Plasmids

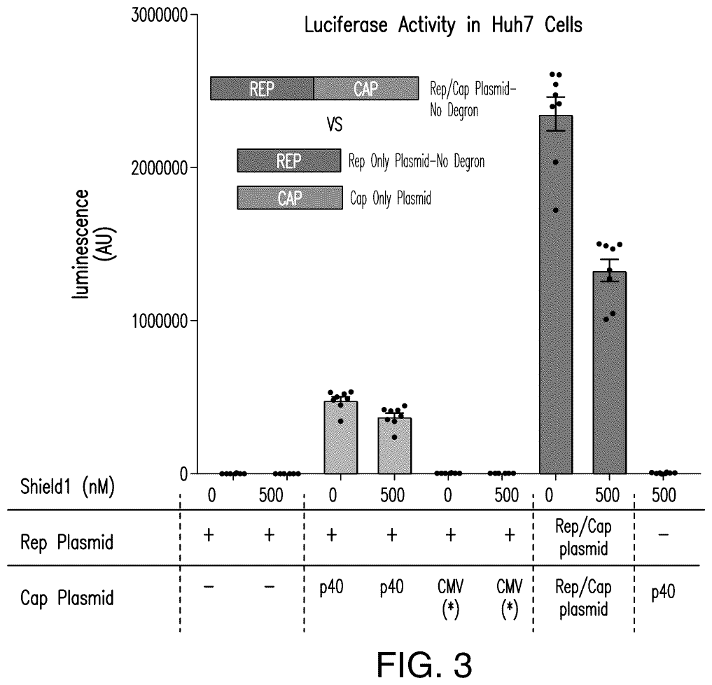

The methods of Example 1, except as specifically noted, were used to determine if rAAV can still be produced by transfection of Rep and Cap on separate plasmids. A Rep/Cap-plasmid without degron, a Rep-only plasmid without degron, and a Cap-only plasmid were tested. The Rep was under the control of a CMV promoter. For Cap, a CMV promoter (CMV-Cap (*), where the end of Rep is missing) and a P40 promoter (P40-Cap where the end of Rep is present) were used. As shown in FIG. 3, P40-Cap can rescue rAAV production, as determined by the target cell transduction. However, CMV-Cap cannot rescue rAAV production. FIG. 3 shows that rAAV can be produced by transfection of Rep and Cap on separate plasmids.

### Example 3: Regulation of rAAV Production by the Addition of Shield1

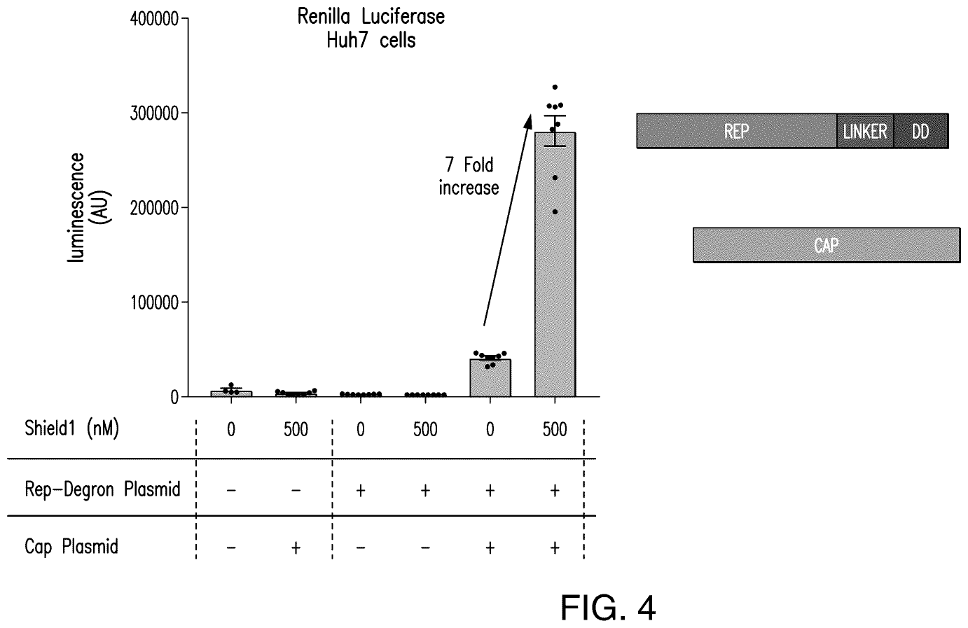

The methods of Example 1, except as specifically noted, were used to determine whether addition of Shield1 can exert post-translational control over accumulation of Rep-Degron. Constructs were transfected to HEK293 cells with or without Cap plasmid (FIG. 4). Shield1 addition rescued rAAV production and transduction of the target cells. As shown in FIG. 4, a Rep-only plasmid C-terminal degron was used. The transduction efficiency was ˜15-20% of the wt Rep/Cap plasmid.

### Example 4: Regulation of Various Rep Constructs

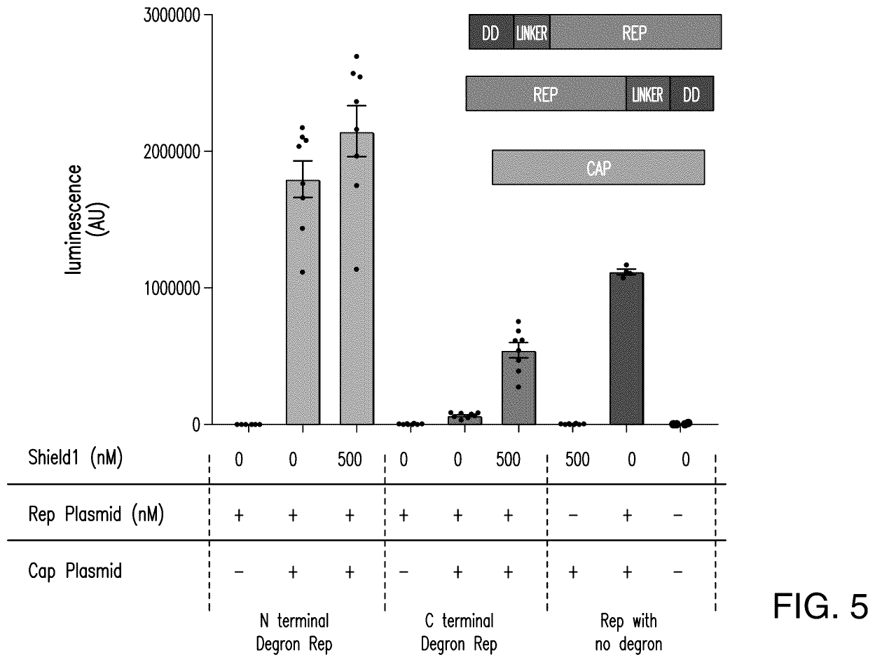

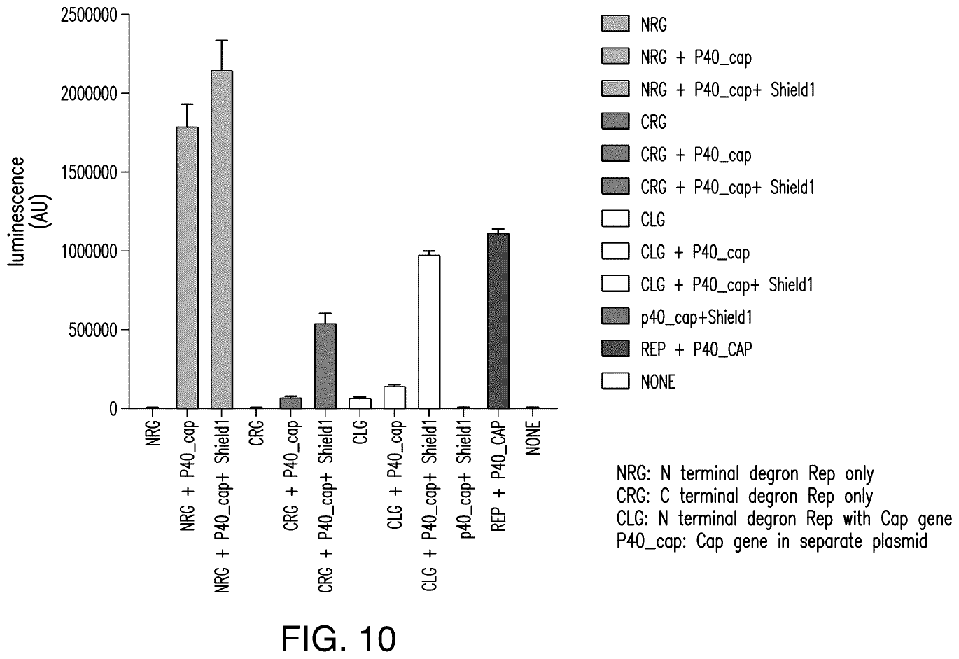

The methods of Example 1, except as specifically noted, were used to determine whether the location of the degron (i.e., a C-terminally or N-terminally located degron) impacts the post-translational regulation of Rep expression. Huh7 cells were transduced with supernatant from HEK293 cells as described above. A P40-Cap plasmid was used to drive expression of Cap. As shown in FIGS. 5 and 10, rAAV production from Rep constructs having an N-terminally linked degron exhibit less tightly regulated expression as compared to Rep constructs having a C-terminally linked degron.

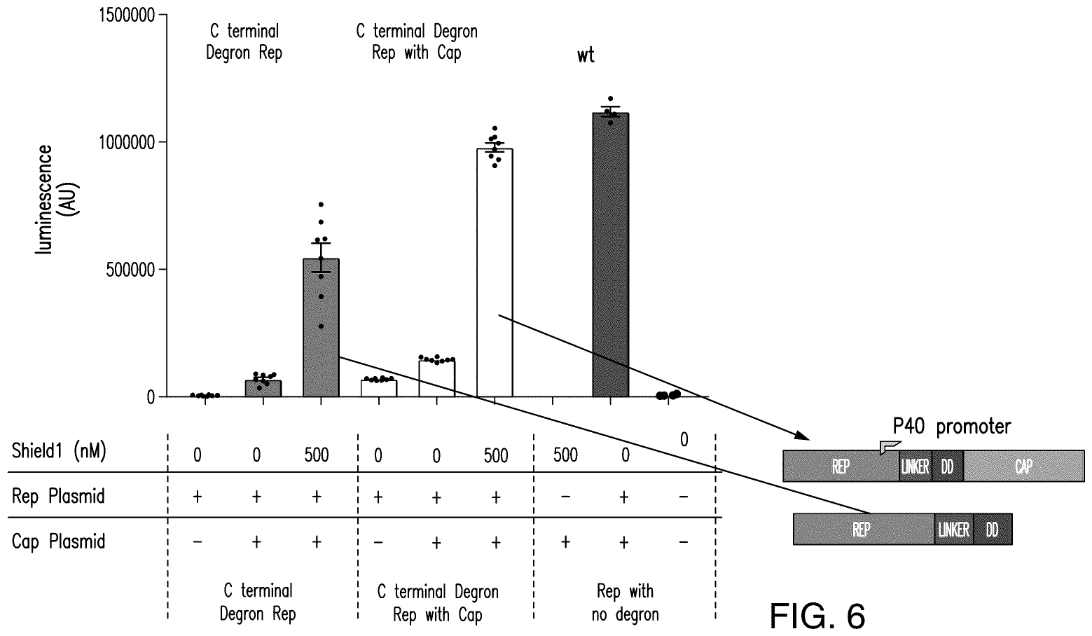

The methods of Example 1, except as specifically noted, were used to determine the effect of Shield1 on post-translational regulation of Rep expression in connection with constructs comprising both Rep and Cap coding sequences relative to constructs having Rep coding sequences only. P40-Cap plasmid was used to drive Cap expression where “Cap Plasmid” was provided (as noted by a “+”). As shown in FIG. 6, despite a modest increase in rAAV production when Rep with a C-terminally linked degron is expressed in the presence of Cap (either expressed from a single Rep/Cap construct or where Cap is expressed in trans), rAAV production is significantly increased when Rep with a C-terminally linked degron is expressed in the presence of both Cap and Shield1. Again, Cap can be expressed from a single Rep/Cap construct or where Cap is expressed in trans.

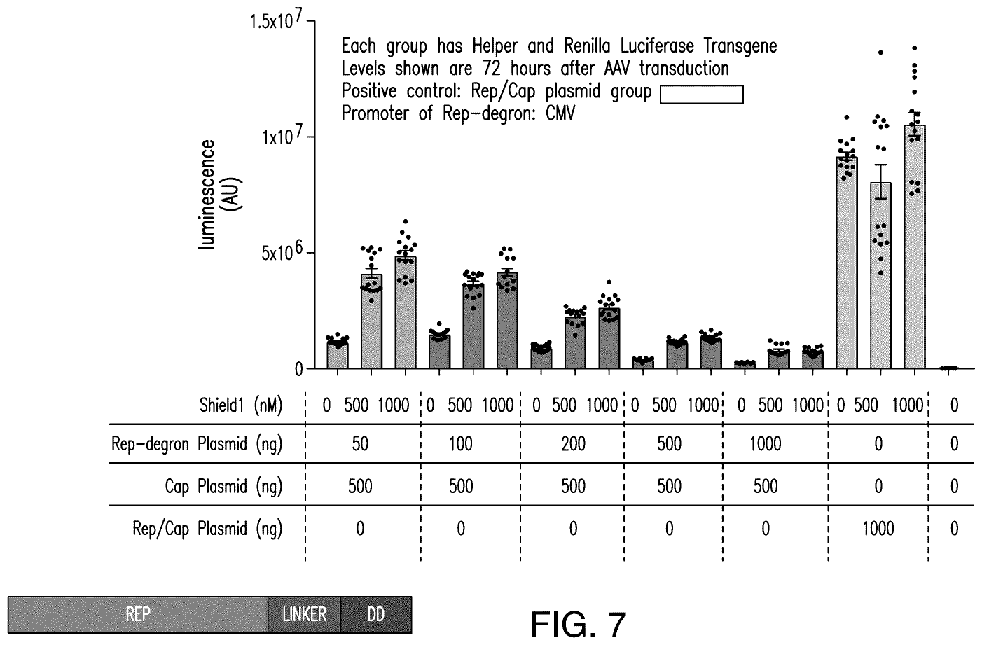

The methods of Example 1, except as specifically noted, were used to determine the effect on rAAV production of adding increasing amounts of plasmid encoding Rep with a C-terminally linked degron. As shown in FIG. 7, increasing the amount of plasmid encoding Rep with a C-terminally linked degron reduces overall rAAV production and leads to less differentiated regulation by Shield1.

### Example 5: Regulation of Rep Constructs with Rigid or Flexible Linkers

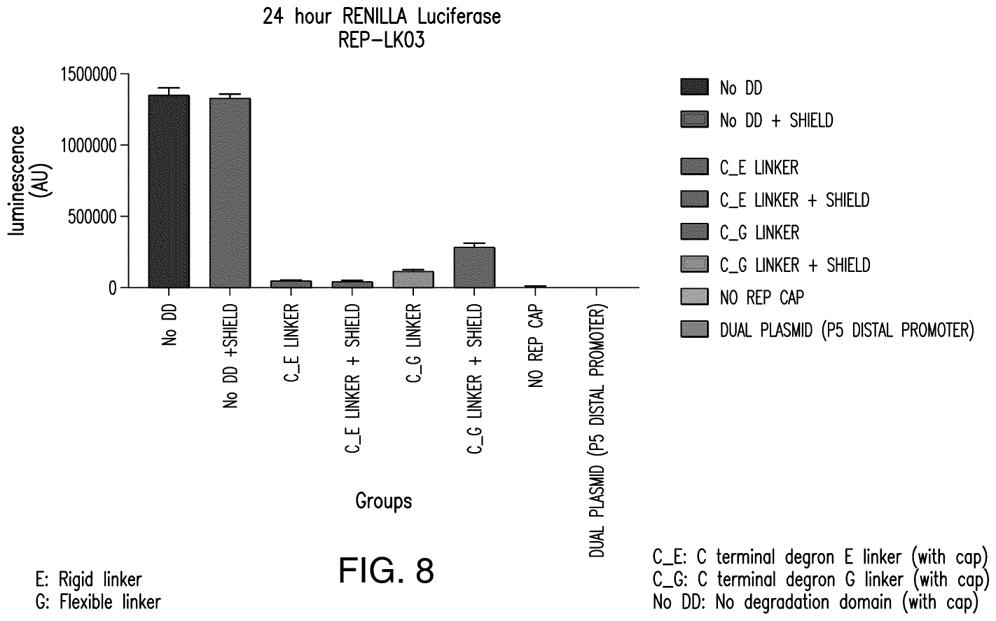

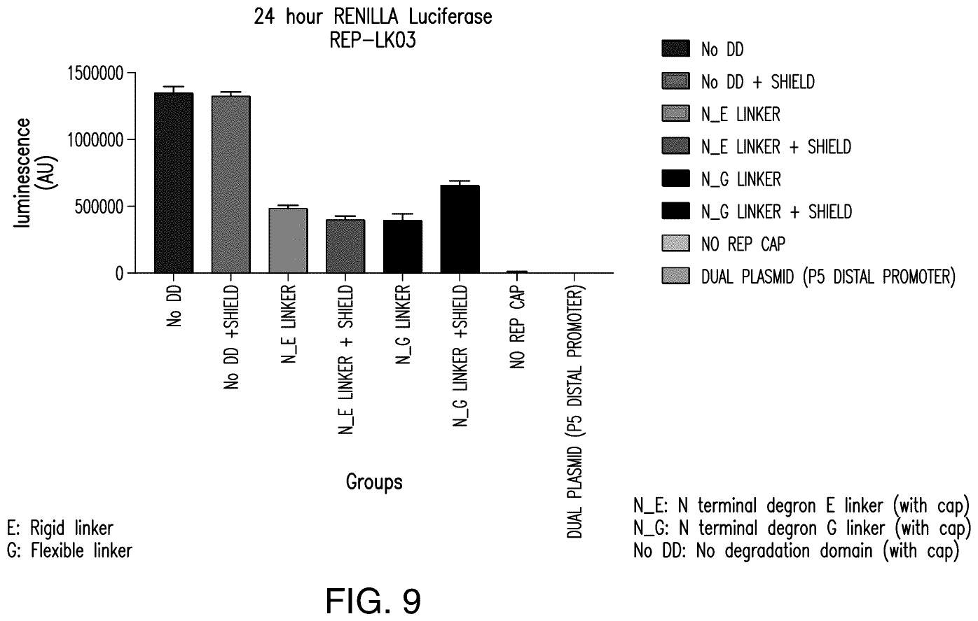

The methods of Example 1, except as specifically noted, were used to determine the effect of linker type between the C-terminus of Rep and the degron. Two types of linkers were tested; a rigid and a flexible linker FIG. 8 shows the results for: Rep lacking a degron (+/−Shield1); Rep with a rigidly linked C-terminal degron (+/−Shield1); Rep with a flexibly linked C-terminal degron (+/−Shield1); and controls where no Rep or Cap was provided and a dual Rep/Cap plasmid (where expression is driven by the P5 distal promoter). FIG. 9 shows the results for: Rep lacking a degron (+/−Shield1); Rep with a rigidly linked N-terminal degron (+/−Shield1); Rep with a flexibly linked N-terminal degron (+/−Shield1); and controls where no Rep or Cap was provided and a dual Rep/Cap plasmid (where expression is driven by the P5 distal promoter). In both FIGS. 8 and 9 use of a flexible linker is associated with Shield1-mediated post-translational control of expression of Rep and resulting rAAV production.

### Example 6: Analyses of Rep Expression & AAV Production

A. Materials & Methods

a. Transfection

Expi293 cells were seeded 1.4E6 viable cells/mL in Expi293 media the day before transfection. Transfection was performed when the viable cells were between 2E6-3E6 cells/mL. PEI pro transfection reagent (Polyplus), OptiMEM (Gibco) were used. PEI:DNA ratio was 2 and 0.6 μg DNA per million cells was used. Triple transfections were performed with RHM4-1 Rep Cap, Helper and Gaussia luciferase plasmids. Split Rep and Cap plasmids were used in a ratio of 1:4 (Rep:Cap). In some cases split Rep Cap plasmids and TIR1 or CMV-Tet were used in a ratio of 1:3:1 (Rep:Cap:TIR1 or CMV-Tet). 1.5 mM SAHA was added after the addition of DNA-PEI complexes. In some cases next day Shield1 or TMP or Auxin and/or Doxycycline was added. For Doxycycline treated samples another treatment of Dox was performed 24 hr later than first treatment. Cells harvested at 4,8,24 hr post-treatment and 44 hr or 68 hr or 72 hr post-transfection; centrifuged and washed with cold PBS. These cell pellets were kept at −80 freezer. 1 mL of cells harvested 44 hr or 68 hr or 72 hr post-transfection for qPCR.

b. Western Blot

Cell pellets were lysed with RIPA buffer (Pierce) including protease and phosphatase inhibitors and EDTA on ice for 30 minutes. Lysates were centrifuged max speed for 15 minutes and supernatant was transferred to a new Eppendorf tube and used for Western. Protein concentrations were measured with BCA kit (Pierce). 30 lag protein per lane loaded for SDS-PAGE gels. These gels were transferred to either PVDF or Nitrocellulose membrane. Membranes were blocked with blocking buffer (Li-Cor) for 30 minutes at room temperature. Anti-AAV Rep clone 303.9 (ARP, cat #03-61069) purified mouse monoclonal antibody was diluted 500 times in either blocking buffer (Li-Cor) or antibody diluent (Li-Cor). Membranes were incubated overnight in cold room either rocking or non-rocking. Next day membranes were washed 3 times 10 minutes each with 1×TBST buffer (Invitrogen). Goat anti-mouse Alexa Flour 680 secondary antibody was diluted 1000 times in either blocking buffer (Li-Cor) or antibody diluent (Li-Cor). Membranes were incubated at room temperature rocking for 45 minutes. Then membranes were washed 6 times 10 minutes each with 1×TBST buffer. Membranes were scanned with Odyssey (Li-Cor). Anti-GAPDH rabbit antibody (Cell Signaling) was diluted 5000 times in either blocking buffer (Li-Cor) or antibody diluent (Li-Cor) as a loading control. Primary incubation was for 1 hour at room temperature rocking. Goat anti-rabbit Alexa Flour 800 secondary antibody was diluted 10000 times in either blocking buffer (Li-Cor) or antibody diluent (Li-Cor). c. qPCR

Frozen harvested cells were thawed and sonicated. 300 μL sonicated cells were treated with Benzonase for 1 hour at 37° C. incubator on a rotator/shaker. Then DNaseI treatment was performed for 15 minutes. Reaction was stopped with stop reagent (0.2% SDS/5 mM EDTA/0.2M NaCl), heated at 95° C. for 10 minutes and centrifuged. Serial dilutions were performed to dilute 10000 or 100000 times. Taqman qPCR was performed with specific primers and probe using Quant Studio Real-Time PCR machine.

B. FKBP Degron Tagged Rep Protein Expression & AAV Production

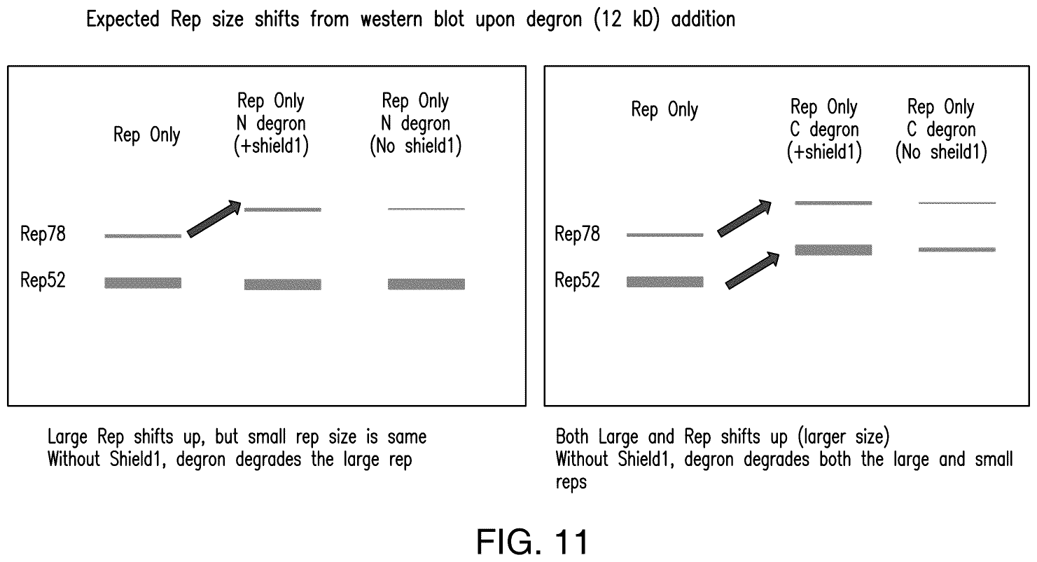

In the instant study, the regulation of AAV production via Rep proteins with an FKBP derived degron was investigated. As a preliminary matter, FIG. 11 illustrates the molecular weight changes of the Rep protein upon tagging with the degron. Specifically, FIG. 11 describes the possible size changes in a western blot after the addition of a degron tag to the N-terminal or C-terminal of Rep protein. Addition of degron to the N-terminal of Rep protein (N-degron) only changes the Molecular Weight of the large Rep but the small Rep protein size is unchanged. Small Rep Molecular Weight is not affected by an N-terminal protein tag because p19 promoter, which drives the expression of small Rep, located within the Rep gene. Small Rep protein does not share the same N-terminal sequence as the large Rep protein. In contrast, addition of a degron to C-terminal of Rep protein (C-degron) changes the Molecular Weights of both small and large Rep proteins as C terminal is same for both. Addition of small molecule such as Shield1 or TMP to the cell culture media can inhibit the protein degradation and the band intensity will change accordingly. Example shown here is FKBP derived degron

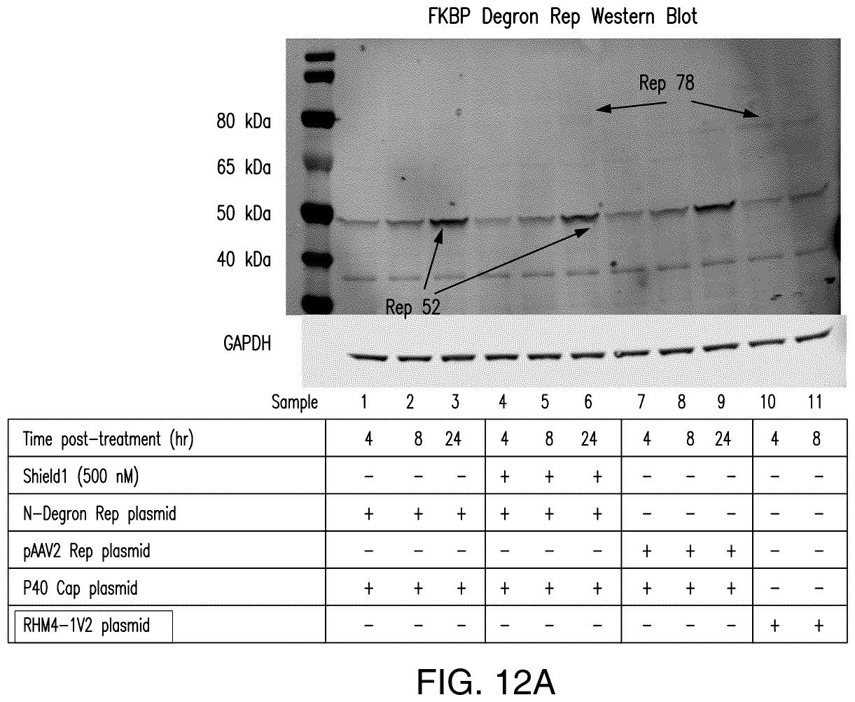

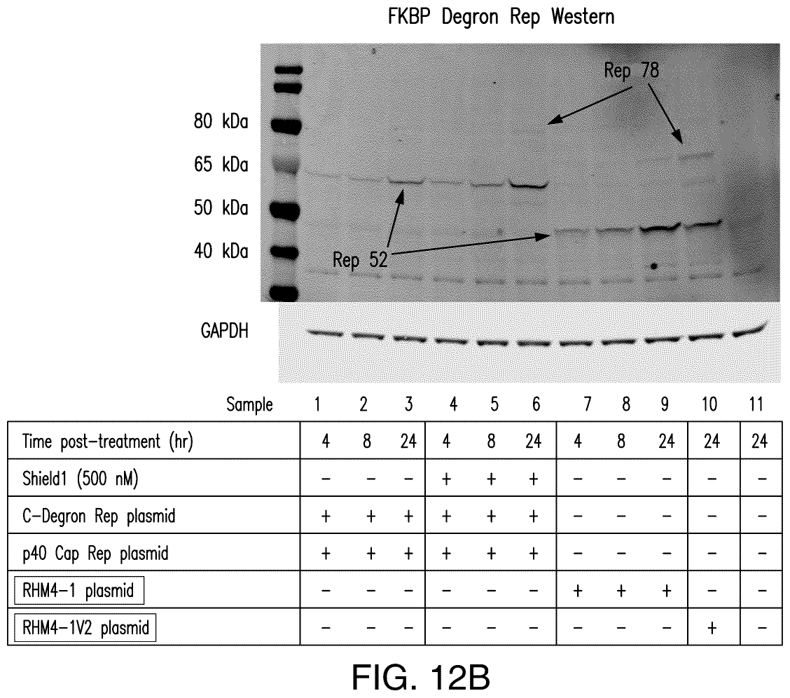

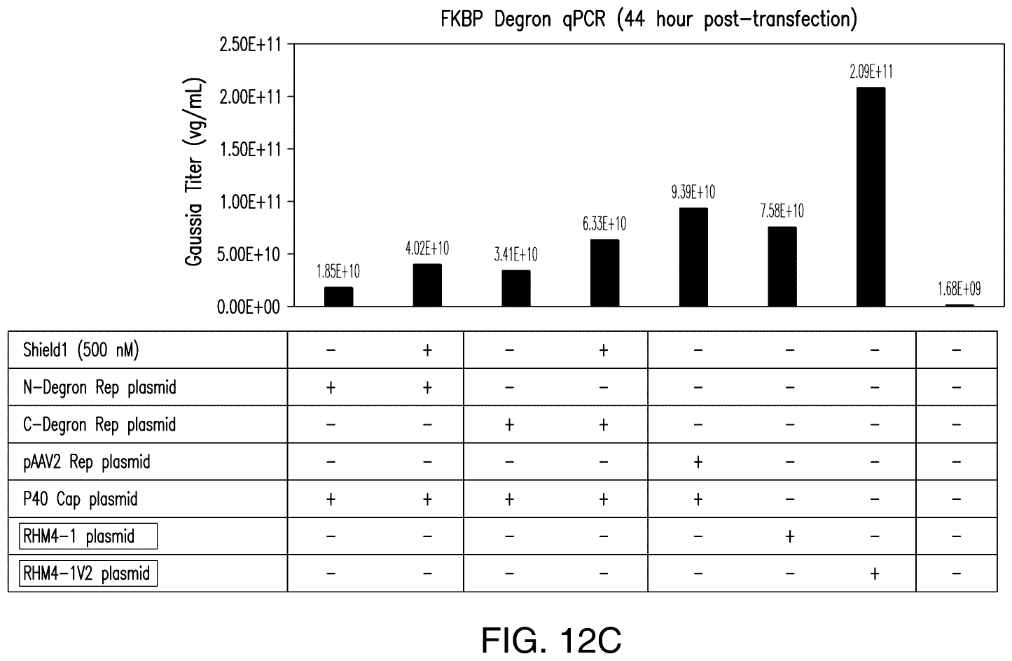

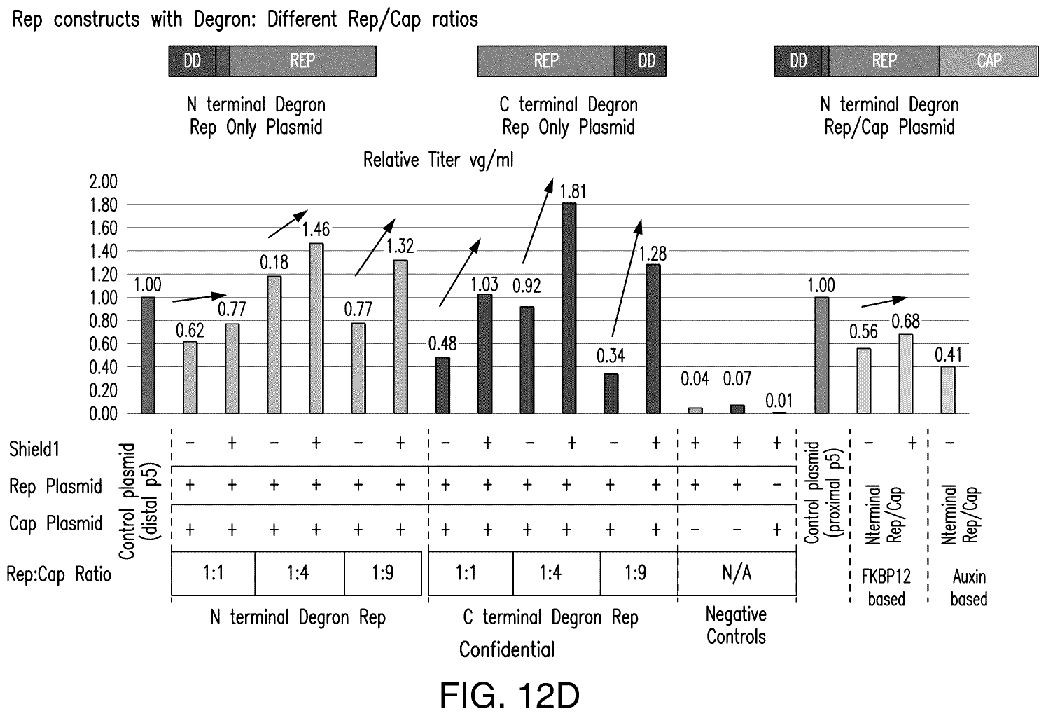

In this study, RHM4-1 was used as the AAV capsid in accordance with the Materials & Methods presented in Section 6A, above. As depicted in FIGS. 12A-12D “RHM4-1” and “RHM4-1v2” are the plasmids that express both Rep proteins with no degrons and RHM4-1 Cap. In contrast, “p40Cap plasmid” only expresses RHM4-1 Capsid, while “pAAV2 Rep plasmid” expresses Rep proteins from AAV2. The ITR plasmid used in these studies has Gaussia Luciferase as transgene.

FIG. 12A depicts a western blot of N-terminal FKBP degron tagged Rep proteins. In this study a plasmid expressing N-terminal degron containing Rep (N-degron Rep) construct was used for AAV production. The samples were collected 4, 8, or 24 hours after Shield1 addition to the media. Molecular Weight and expression of small Rep (Rep52) are not affected by degron presence or absence as explained in FIG. 11 (Samples 1-6 vs Samples 7-11). The control samples produced using Rep with no degron are loaded in lanes 7-11. Due to the inherent plasmid design, large Rep (Rep78) expression is much lower than the small Rep expression in all samples. Large Rep expression from N-degron Rep samples increases after the addition of Shield1 (Samples 4-6) compared to the untreated controls (Samples 1-3). Molecular Weight of large Rep protein is greater for the samples with N-degron Rep (Samples 1-6 vs 7-11), but no change for the small Rep Molecular Weight is observed for these samples as explained at FIG. 11.

FIG. 12B depicts a western blot of C-terminal FKBP degron tagged Rep proteins. In this study a plasmid expressing C-terminal degron containing Rep construct (C-degron Rep) was used for AAV production and the samples were collected at different time points (4, 8, and 24 hour) following Shield1 induction. Small and large Rep expression is higher with Shield1 (Samples 4-6) than the untreated controls (Samples 1-3) at the same time points. Small and large Rep Molecular Weights are greater with C-terminal FKBP degron and a shift is observed compared to the samples with no degron containing Rep proteins (Samples 1-6 vs 7-11).

FIG. 12C depicts the AAV titers of the samples from FIGS. 12A and 12B. AAV titer increases upon addition of the Shield1 molecule to the media used for AAV production. In this study the cells were transfected with Rep-degron containing plasmids along with the other plasmids required for AA production. Shield1 molecule addition increases AAV titer for N- and C-terminal degron constructs compared to the untreated controls.

FIG. 12D depicts the AAV titers observed with Rep and Cap plasmids transfected at different ratios. In this study the degron tagged Rep proteins are regulated in cells transfected with different ratios of Rep and Cap plasmids. As illustrated in the results, AAV production can be regulated via Degron tagged Rep proteins provided as sperate plasmids transfected at different ratios to the HEK293 cells as listed at the bottom of the Figure. The titers were normalized against the titer from the control transfection of using Rep/Cap plasmid with no degron. 500 nM Shield1 was added to the samples to inhibit Rep degradation.

C. ecDHFR Degron Tagged Rep Protein Expression & AAV Production

In the instant study, the regulation of AAV production via Rep proteins with an ecDHFR derived degron was investigated. The ecDHFR derived degron has the following amino acid sequence:

MISLIAALAVDYVIGMENAMPWNLPADLAWFKRNTLNKPVIMGRHTWES
IGRPLPGRKNIILSSQPGTDDRVTWVKSVDEAIAACGDVPEIMVIGGGR
VIEQFLPKAQKLYLTHIDAEVEGDTHFPDYEPDDWESVFSEFHDADAQN
SHSYCFEILERR.

The ecDHFR derived degron has the following DNA sequence:

ATGATCAGTCTGATTGCGGCGTTAGCGGTAGATTACGTTATCGGCATGG
AAAACGCCATGCCGTGGAACCTGCCTGCCGATCTCGCCTGGTTTAAACG
CAACACCTTAAATAAACCCGTGATTATGGGCCGCCATACCTGGGAATC
AATCGGTCGTCCGTTGCCAGGACGCAAAAATATTATCCTCAGCAGTCA
ACCGGGTACGGACGATCGCGTAACGTGGGTGAAGTCGGTGGATGAAGC
CATCGCGGCGTGTGGTGACGTACCAGAAATCATGGTGATTGGCGGCGG
TCGCGTTATTGAACAGTTCTTGCCAAAAGCGCAAAAACTGTATCTGACG
CATATCGACGCAGAAGTGGAAGGCGACACCCATTTCCCGGATTACGAG
CCGGATGACTGGGAATCGGTATTCAGCGAATTCCACGATGCTGATGCG
CAGAACTCTCACAGCTATTGCTTTGAGATTCTGGAGCGGCGGTAA

In this study, RHM4-1 was used as the AAV capsid in accordance with the Materials & Methods presented in Section 6A, above.

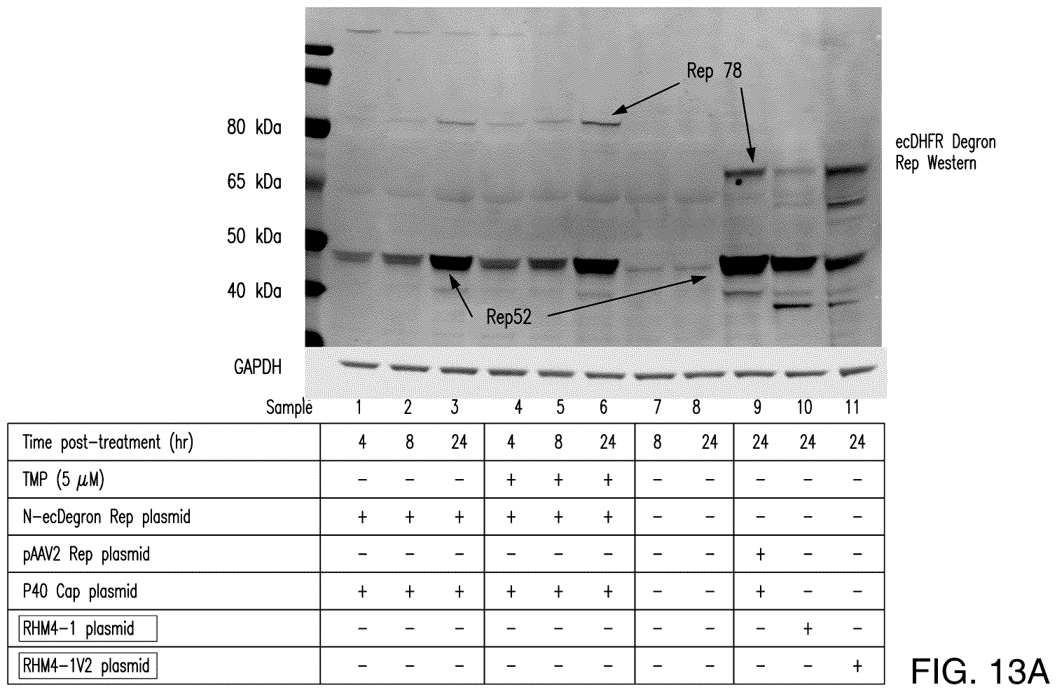

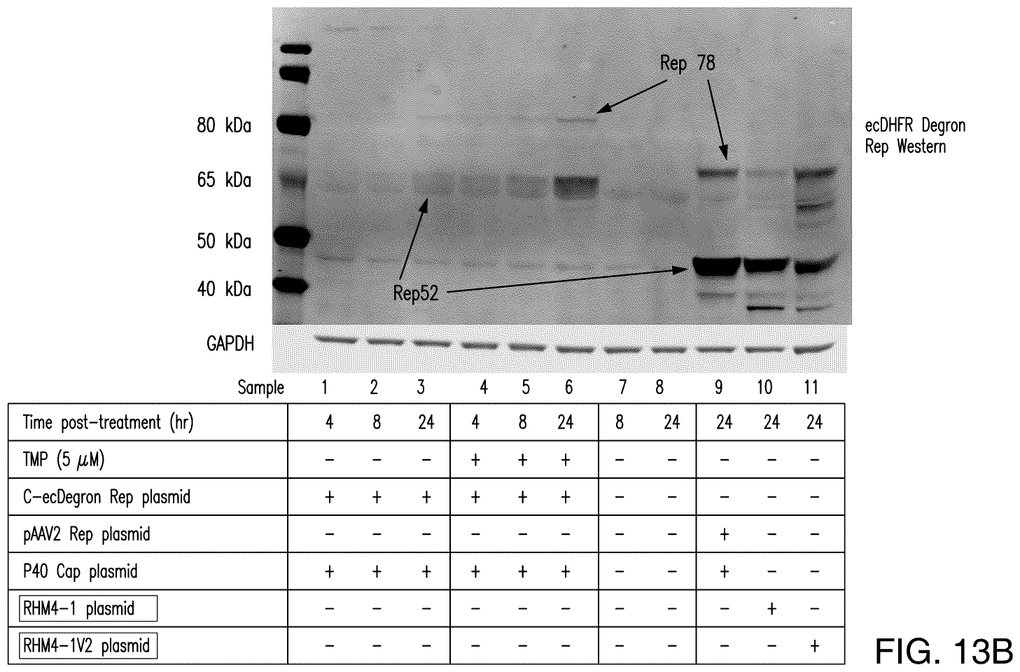

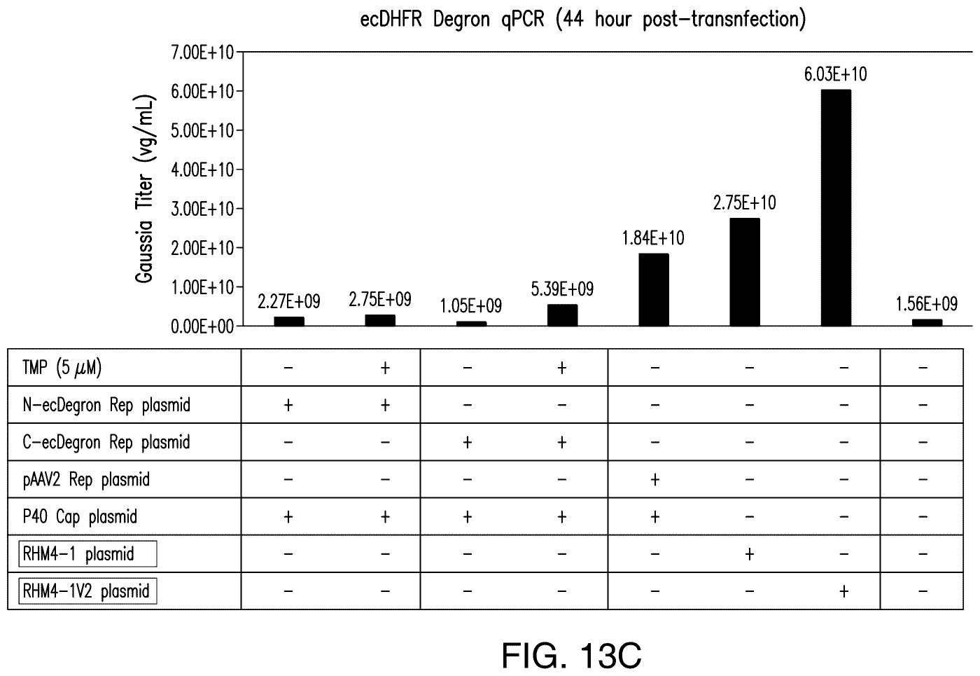

FIG. 13A depicts a western blot of N-terminal ecDHFR Degron tagged Rep protein. In this study N-terminal ecDegron containing Rep (N-ecDegron Rep) construct was used for AAV production and Cap gene is provided as a separate plasmid. Small Rep expression is not affected by N-terminal ecDegron fusion (Samples 1-6) as explained at FIG. 11. Large Rep expression is higher for samples treated with 5 μM TMP post 24 hour time point (Sample 3 vs Sample 6). Large Rep Molecular Weight is bigger as it's tagged with ecDHFR degron (N-terminal) and a shift is observed for those samples (Samples 1-6). As predicted, no such shift is observed for Small Rep proteins as its Molecular Weight remains the same.

FIG. 13B depicts a western blot of C-terminal ecDHFR Degron tagged Rep proteins. In this study C-terminal ecDegron containing Rep (C-ecDegron Rep) construct was used for AAV production and Cap gene is provided as a separate plasmid. Small and large Rep expression is more with 5 μM TMP (Sample 3 vs Sample 6). Small and large Rep Molecular Weights are larger with C-terminal ecDHFR degron (Samples 1-6) and shifts are observed for both proteins.

FIG. 13C depicts the AAV titers of the samples from FIGS. 13a and 13b. Titer levels of the AAV produced via plasmids with ecDHFR Degron containing Rep were analyzed. TMP addition increases AAV titer of the C-terminal degron constructs correlating with the western blot data for Rep expression.

D. Auxin or ecDHFR Degron Tagged Rep Protein Expression & AAV Production

In the instant study, the regulation of AAV production via Rep proteins with either an auxin derived degron or ecDHFR derived degron was investigated. The auxin derived degron has the following amino acid sequence:

MGSVELNLRETELCLGLPGGDTVAPVTGNKRGFSETVDLKLNLNNEPANK
EGSTTHDVVTFDSKEKSACPKDPAKPPAKAQVVGWPPVRSYRKNVMVSC
QKSSGGPEAAAFVKVSLDGAPYLRKIDLRLYKSYDELSNALSNLFSSFT.

The auxin derived degron has the following DNA sequence:

ATGGGCAGTGTCGAGCTGAATCTGAGGGAGACTGAGCTGTGTCTTGGT
CTTCCCGGTGGAGATACAGTGGCTCCGGTAACCGGAAACAAGAGAGGG
TTCTCAGAGACGGTTGATCTGAAGCTAAATCTGAATAATGAGCCTGCA
AACAAGGAAGGATCTACGACTCATGACGTAGTGACTTTTGATTCCAAG
GAGAAGAGTGCTTGTCCTAAAGATCCAGCCAAACCTCCGGCCAAGGCA
CAAGTTGTGGGATGGCCACCGGTGAGATCATACCGGAAGAACGTGATG
GTTTCCTGCCAAAAATCAAGCGGTGGCCCGGAGGCGGCGGCGTTCGTG
AAGGTATCATTAGACGGAGCACCGTACTTGAGGAAAATCGATTTGAGG
TTATATAAAAGCTACGATGAGCTTTCTAATGCTTTGTCCAACTTATTCA
GCTCTTTTACCTGA

In this study, RHM4-1 was used as the AAV capsid in accordance with the Materials & Methods presented in Section 6A, above.

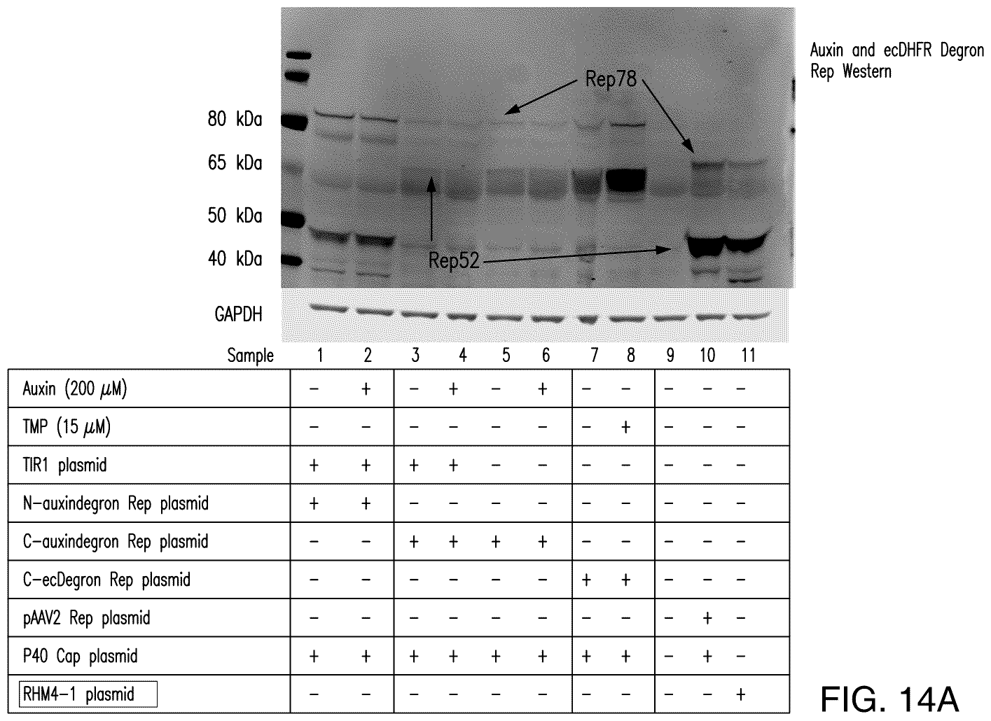

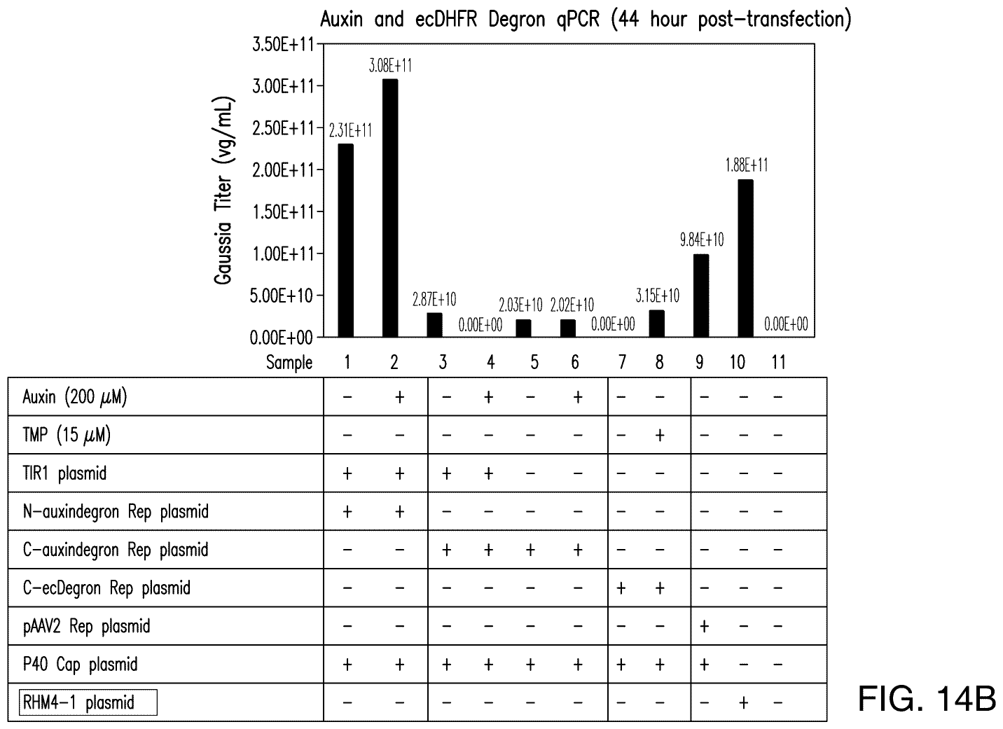

FIG. 14A depicts a western blot of Reps that are tagged with Auxin Inducible degron or ecDHFR degron. Small and Large Rep size are bigger with C-terminal Auxin Inducible Degron containing Rep plasmids (Samples 3-6) and shifts are observed for both proteins. Changes in the Rep expressions for Auxin Inducible degron are hard to observe due to the presence of a nonspecific band with the same size as Degron tagged-Small Rep. This nonspecific band is present even for the untransfected sample (Sample 9). Small and large Rep expression is more with TMP (Samples 7 and 8) for C-terminal tagged ecDHFR degron. Small and large Rep sizes are bigger with C-terminal ecDHFR degron (Samples 7 and 8) and shifts are observed.

FIG. 14B depicts the AAV titers of the samples from FIG. 14A. As illustrated in FIG. 14B, Auxin Inducible Degron and ecDHFR degron can regulate AAV titer where Auxin Inducible degrons function only when the auxin and TIR1 ubiquitin Ligase are present in the cells along with the Auxin dependent degron. The amino acid sequence of the TIR-1 protein is:

MQKRIALSFPEEVLEHVFSFIQLDKDRNSVSLVCKSWYEIERWCRRKVF
IGNCYAVSPATVIRRFPKVRSVELKGKPHFADFNLVPDGWGGYVYPWIE
AMSSSYTWLEEIRLKRMVVTDDCLELIAKSFKNFKVLVLSSCEGFSTDG
LAAIAATCRNLKELDLRESDVDDVSGHWLSHFPDTYTSLVSLNISCLAS
EVSFSALERLVTRCPNLKSLKLNRAVPLEKLATLLQRAPQLEELGTGGY
TAEVRPDVYSGLSVALSGCKELRCLSGFWDAVPAYLPAVYSVCSRLTTL
NLSYATVQSYDLVKLLCQCPKLQRLWVLDYIEDAGLEVLASTCKDLREL
RVFPSEPFVMEPNVALTEQGLVSVSMGCPKLESVLYFCRQMTNAALITI
ARNRPNMTRFRLCIIEPKAPDYLTLEPLDIGFGAIVEHCKDLRRLSLSG
LLTDKVFEYIGTYAKKMEMLSVAFAGDSDLGMHHVLSGCDSLRKLEIRD
CPFGDKALLANASKLETMRSLWMSSCSVSFGACKLLGQKMPKLNVEVID
ERGAPDSRPESCPVERVFIYRTVAGPRFDMPGFVWNMDQDSTMRFSRQI
ITTNGL

Unlike FKBP and ecDHFR dependent degrons which prevent proteins degradation upon small molecule addition, Auxin Inducible degron leads to degradation of the protein it's tagged to. Auxin, TIR1 and degron needs to be present for Auxin Inducible Degron to be functional. The AAV production is undetectable for the sample with all the components (degron, auxin and TIR1) of Auxin mediated degron are present (Sample 4). This shows the functionality of this degron for AAV production regulation at the C-terminal of Rep protein. ecDHFR degron tagged Reps can produce AAV in the presence of TMP molecule (Sample 8) which was undetectable without TMP molecule (Sample 7).

E. Rep Protein Expression & AAV Production In the Presence of Shield1 or dTag13

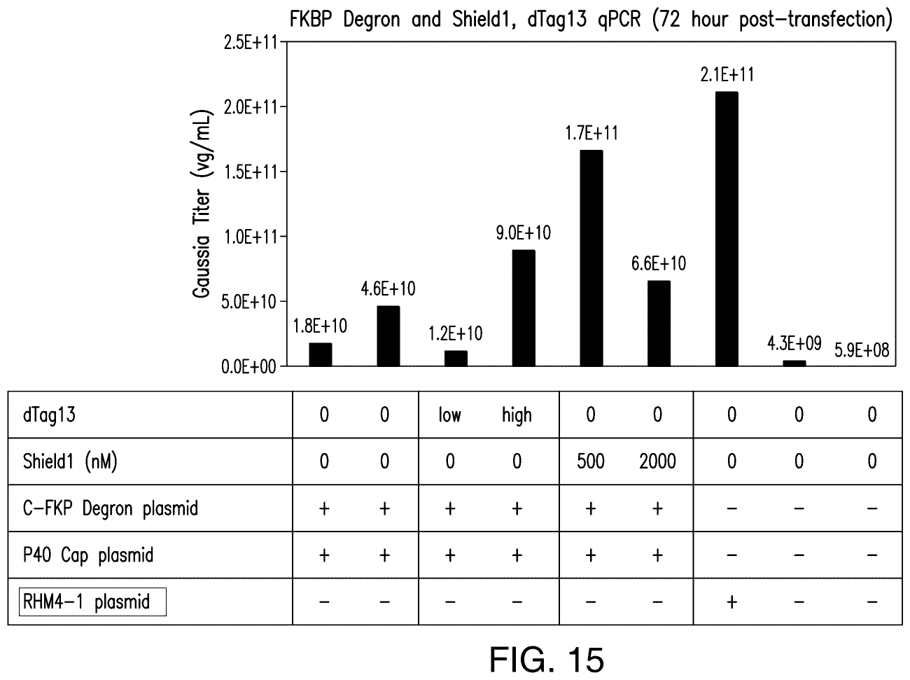

FIG. 15 depicts the impact of different doses of Shield1 and dTag13 on AAV productions. In this study, Rep constructs with C-terminal tagged degron were tested with different doses of Shield1 and dTag13. Different Shield1 levels were compared to see the effect on titer. Shield1 is more effective at 500 nM concentration for increasing the AAV production. Also tested was dTag13, a molecule binds to several FKBP derived domains and further derives their degradation. dTag13 did not decrease the basal level of the AAV production in this study.

F. Rep Protein Expression & AAV Production Under Transcriptional Level Control System and/or Degron Level Control

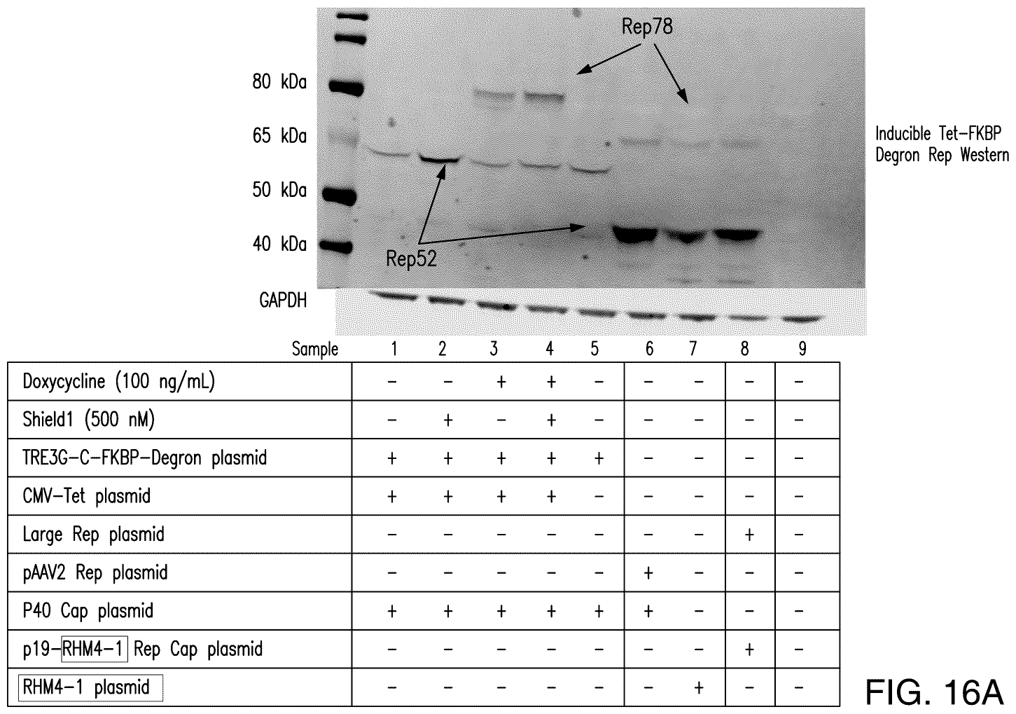

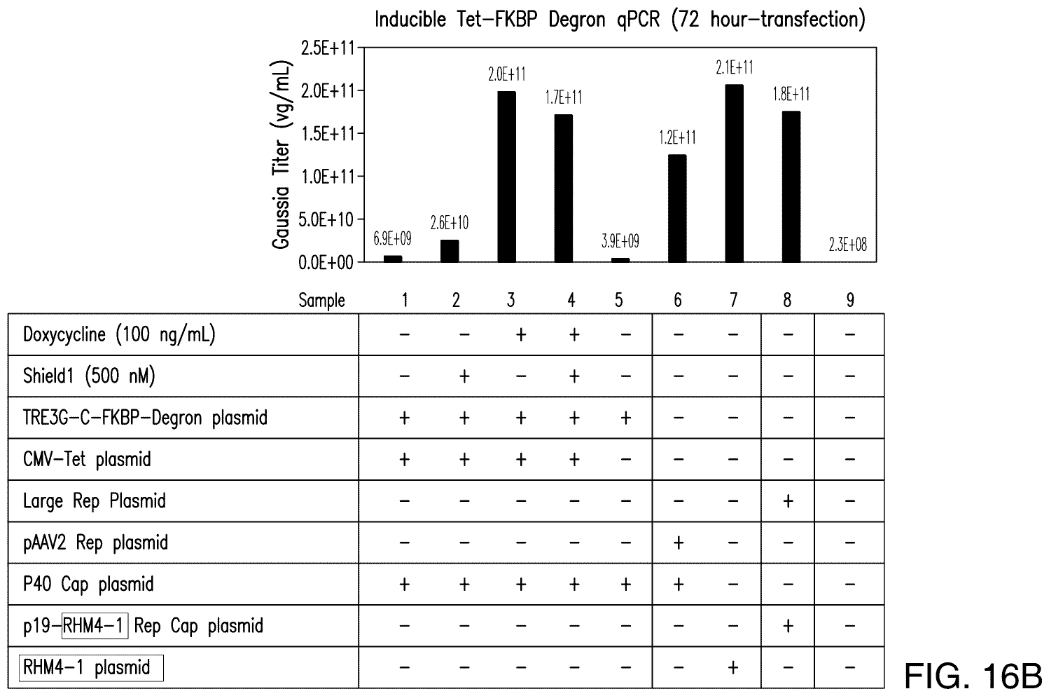

FIG. 16A depicts a western blot of Rep with C-terminal degron under the control of TRE3G promoter. The DNA sequence of the TRE3G promoter is:

GAGTTTACTCCCTATCAGTGATAGAGAACGTATGAAGAGTTTACTCCCT
ATCAGTGATAGAGAACGTATGCAGACTTTACTCCCTATCAGTGATAGA
GAACGTATAAGGAGTTTACTCCCTATCAGTGATAGAGAACGTATGACC
AGTTTACTCCCTATCAGTGATAGAGAACGTATCTACAGTTTACTCCCTA
TCAGTGATAGAGAACGTATATCCAGTTTACTCCCTATCAGTGATAGAGA
ACGTATAAGCTTTAGGCGTGTACGGTGGGCGCCTATAAAAGCAGAGCT
CGTTTAGTGGGCGCGCCACCGTCAGATCGCCTGGAGCAATTCCACAAC
ACTTTTGTCTTATACCAACTTTCCGTACCACTTCCTACCCTCGTAAA

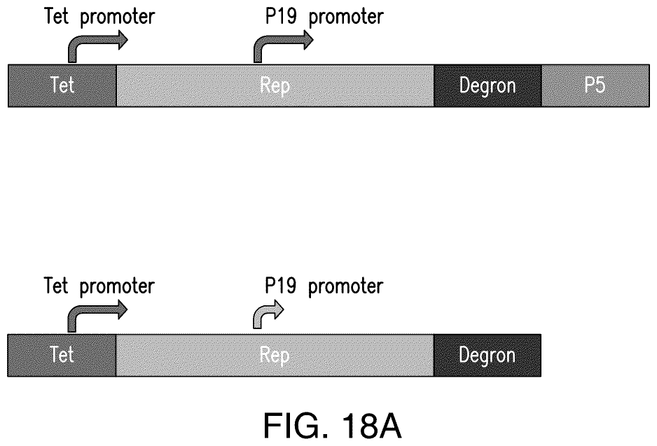

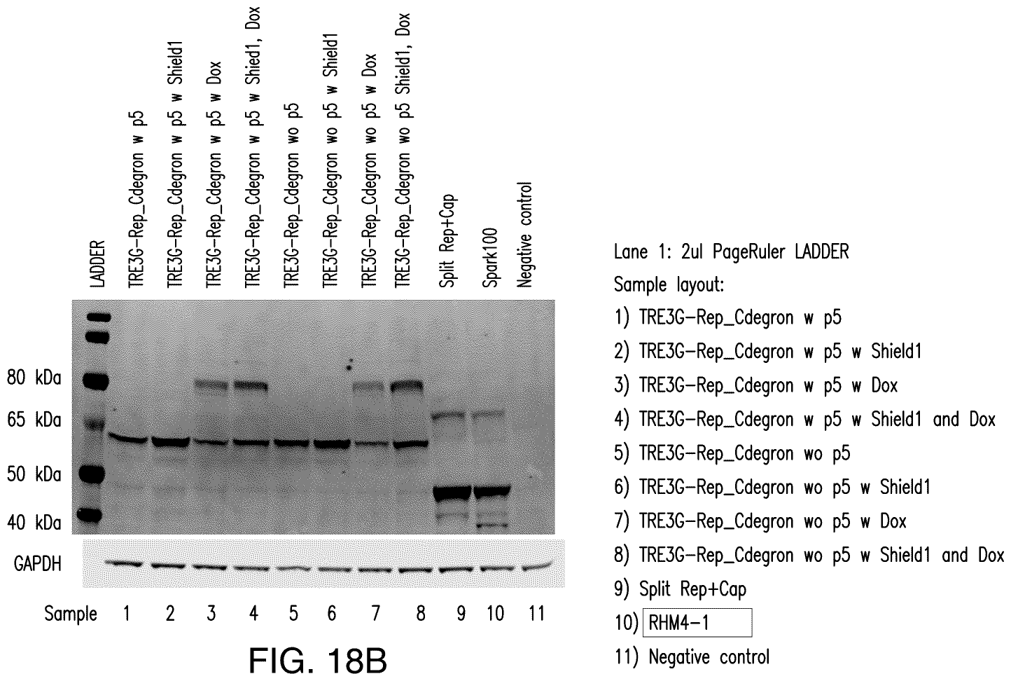

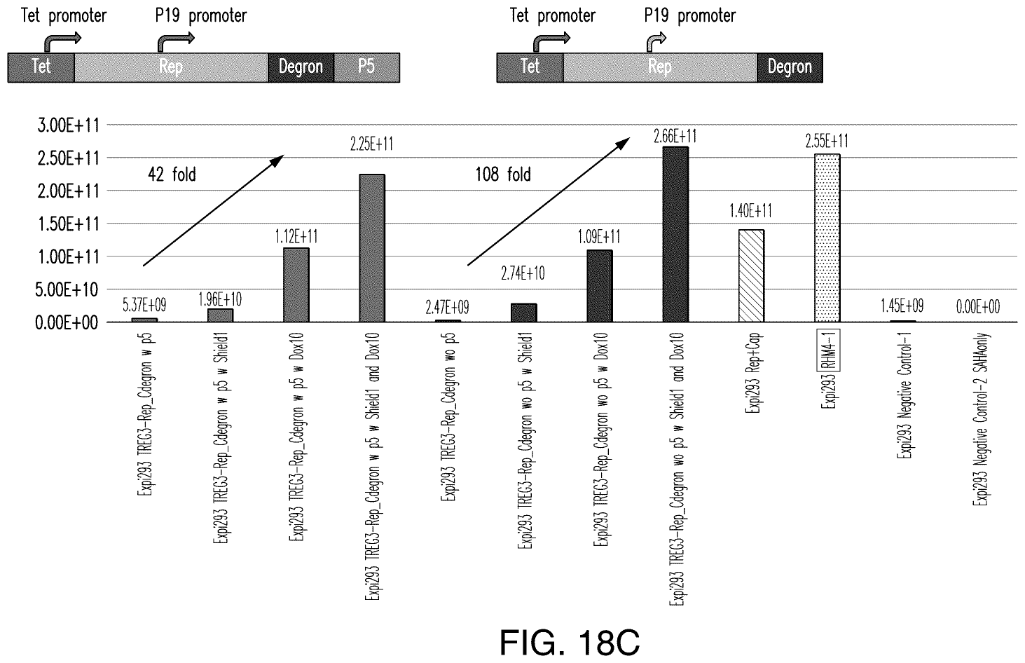

In this study a Tet response element containing promoter (TRE3G) is cloned in front of the Rep gene as shown at FIG. 18A. This promoter can be activated in the presence of Tet proteins and doxycycline induction. Rep with C-terminal Degron under Tet Response Element promoter control is shown to be functional for AAV production regulation. Large Rep is under the control of Tet promoter and small Rep expression is provided by p19 promoter (Samples 1-5). Large Rep expression is tightly controlled by this system and can be detected upon addition of doxycycline (Samples 3 and 4). Shield1 can upregulate small Rep expression (Sample 1 vs Sample 2) and large Rep expression (Sample 3 vs Sample 4). A separate plasmid is used for supplying Tet-On 3G transactivator protein where transactivator protein expression is under the control of ubiquitous CMV promoter (Samples 1-4). The amino acid sequence of the transactivator protein is:

MSRLDKSKVINSALELLNGVGIEGLTTRKLAQKLGVEQPTLYWHVKNKRA
LLDALPIEMLDRHHTHSCPLEGESWQDFLRNNAKSYRCALLSHRDGAKVH
LGTRPTEKQYETLENQLAFLCQQGFSLENALYALSAVGHFTLGCVLEEQE
HQVAKEERETPTTDSMPPLLKQAIELFDROGAEPAFLFGLELIICGLEKQ
LKCESGGPTDALDDFDLDMLPADALDDFDLDMLPADALDDFDLDMLPG

The DNA sequence of the CMV promoter is:

CGATGTACGGGCCAGATATACGCGTTGACATTGATTATTGACTAGTTAT
TAATAGTAATCAATTACGGGGTCATTAGTTCATAGCCCATATATGGAGT
TCCGCGTTACATAACTTACGGTAAATGGCCCGCCTGGCTGACCGCCCAA
CGACCCCCGCCCATTGACGTCAATAATGACGTATGTTCCCATAGTAACG
CCAATAGGGACTTTCCATTGACGTCAATGGGTGGACTATTTACGGTAAA
CTGCCCACTTGGCAGTACATCAAGTGTATCATATGCCAAGTACGCCCCC
TATTGACGTCAATGACGGTAAATGGCCCGCCTGGCATTATGCCCAGTAC
ATGACCTTATGGGACTTTCCTACTTGGCAGTACATCTACGTATTAGTCA
TCGCTATTACCATGGTGATGCGGTTTTGGCAGTACATCAATGGGCGTGG
ATAGCGGTTTGACTCACGGGGATTTCCAAGTCTCCACCCCATTGACGTC
AATGGGAGTTTGTTTTGGCACCAAAATCAACGGGACTTTCCAAAATGTC
GTAACAACTCCGCCCCATTGACGCAAATGGGCGGTAGGCGTGTACGGT
GGGAGGTCTATATAAGCAGAGCTCTCTGGCTAACTAGAGAACCCACTG
CTTACTGGCTTATCGAAATT

FIG. 16B depicts the AAV titers of the samples from FIG. 16A. This study indicates that AAV production can be regulated using C-terminal Degron tagged Rep under the control of TRE3G promoter and that Doxycycline induction can increase the AAV production ˜29 times from the basal levels. The levels of AAV titers from these samples are comparable to the control samples with no degron (Samples 6-8).

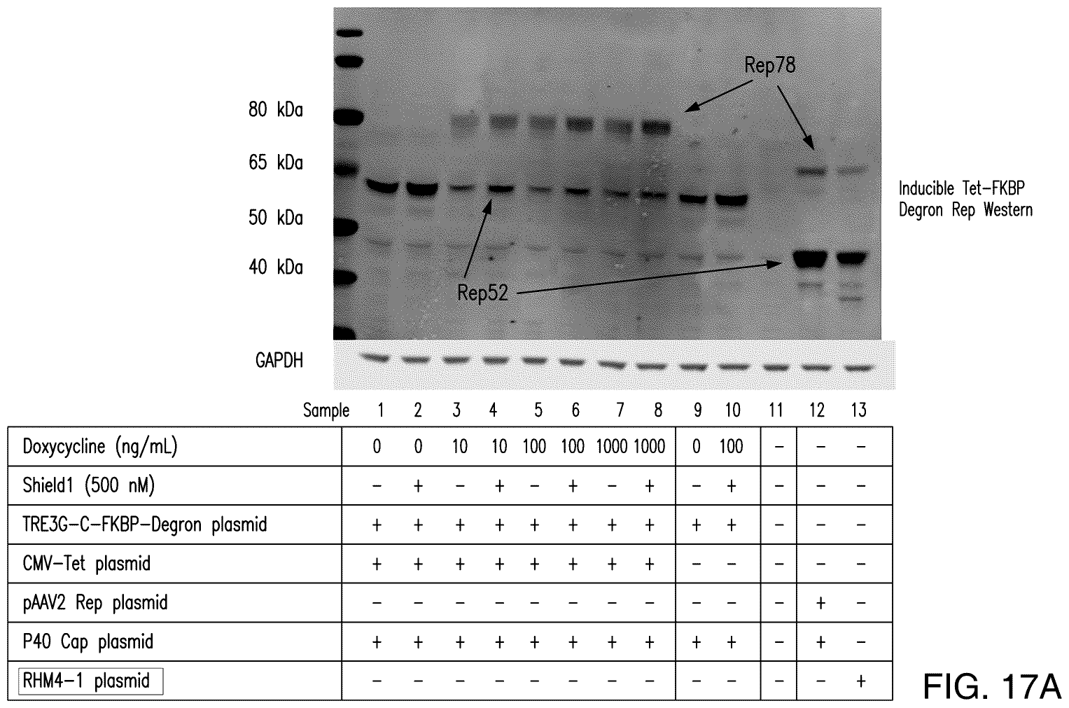

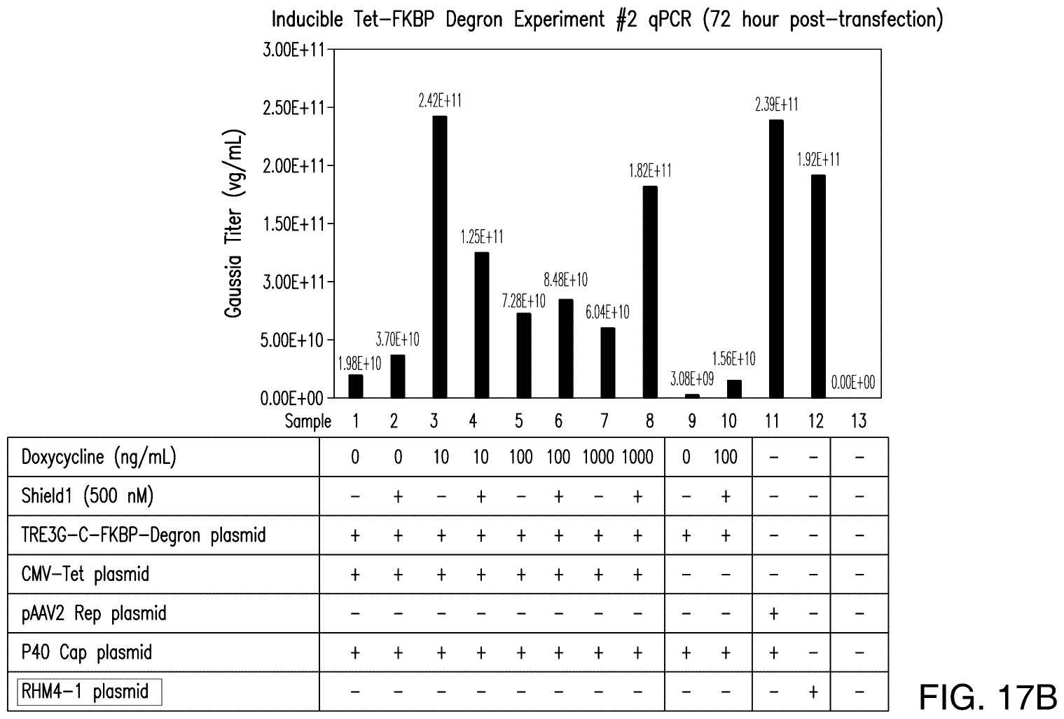

FIG. 17A depicts a western blot of Rep constructs with C-terminal degron. In this study, the effect of different doxycycline levels on AAV production from Rep-degron constructs under TRE3G promoter control was tested. Large Rep is under the control of TRE3G promoter (Samples 1-10). Doxycycline effectively induces the expression large Rep (Samples 3-8). Large and small Rep expression is increased in the presence of Shield1 (Samples 2, 4, 6, and 8 vs Samples 1, 3, 5, and 7). Omission of plasmid that expresses Tet-On 3G transactivator protein from the transfection leads to inactivation of doxycycline induction (Sample 9 and Sample 10).

FIG. 17B depicts the AAV titers of the samples from FIG. 17A. This study illustrates that Rep under the control of TRE3G and degron can produce comparable AAV amount to positive control, unregulated Rep/Cap plasmid. Different doxycycline levels can increase the AAV titer with or without Shield1 molecule, but the basal level is lower in the absence of Shield (Sample1) as a proof for tighter regulation with dual system.

FIG. 18A depicts the effect of a p5 promoter on Rep protein levels in TRE3G-Rep-Degron system. In this study plasmids with degron tagged Rep constructs under the control of a TRE3G promoter were produced with or without p5 promoter at the 3′ of the Rep Gene.

FIG. 18B depicts a western blot of Rep constructs with C-terminal degron and TRE3G promoter. In this study Large Rep is under the control of a TRE3G promoter (Samples 1-8). Doxycycline effectively induces the expression large Rep (Sample 3-4 and 7-8) and large Rep expression is increased further with Shield1 addition (Sample 4 and Sample 8). As illustrated in the Figure, both plasmids (with or without p5 promoter at the 3′ of Rep gene) can express Large and Small Reps.

FIG. 18C depicts the AAV titers of the samples from FIG. 18B. This study indicates that a P5 promoter may not be essential for AAV production in transient transfection using TRE3G system. As illustrated in the Figure, both plasmids can produce comparable levels of AAV titers. Again, Gaussia Luciferase was used as the gene of interest (GOI)

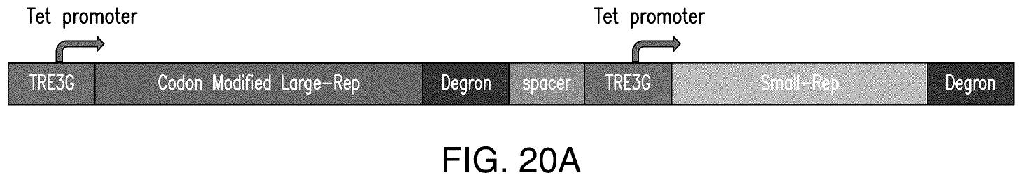

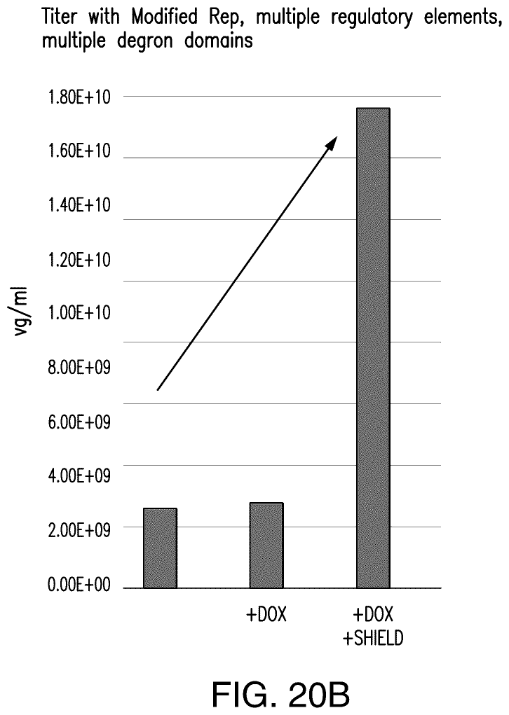

FIGS. 20A-20B depict that codon-modified Rep with degron motif and indicate that such constructs can be used for AAV production. The Figures also show a second AAV rep gene, small-Rep, under a regulatory element and degron domain. The results achieved with these constructs demonstrate that the disclosed method can be customized to work with multiple regulatory elements, whether they are the same or different, multiple degron domains, whether they are the same or different, and modified or unmodified genes encoding AAV proteins.

### Example 7: Degron Tagged Helper Genes for Regulated AAV Production

As illustrated in this example, degron tagged Helper genes can be used for regulating AAV production. The classical helper plasmid used for AAV production comprises E2A, E4 and VA genes. In this example, open reading frames from E2A, E4 and VA genes were cloned into separate plasmids. The selected open reading frames for E2A, E4 and VA are DBP, E4-E34K and VA2 respectively. The triple transfection method described in Examples 1-6 was modified to allow E2A-DBP, E4-E34K or VA2 genes to be supplied in separate plasmids instead of a single Helper plasmid for use in triple production.

A. Materials & Methods

For the instant degron studies utilizing helper genes, the DNA Binding Protein (DBP), E4-E34K gene and VA2 were selected respectively as the open reading frames for E2A, E4 and VA genes. These selected open reading frames from the E2A, E4 and VA genes were cloned into separate plasmids under the control of CMV promoter. An FKBP derived degron was cloned into the C-terminal of the E2A-DBP gene.

The triple transfection method was modified to allow E2A-DBP, E4-E34K and VA2 genes to be supplied in three separate plasmids instead of a single Helper plasmid. For transfection and cell lysis same reagents and methods were used as Rep-degron experiments described herein. The molar ratio for the plasmids used for quintuple transfection (ITR-GOI: Rep/Cap: DBP: E34K: VA2) with separate Helper plasmids was 2:2:1:1:1. The molar ratios for the plasmids used for triple transfection (ITR-GOI: Rep/Cap: Helper) with single Helper plasmid was 2:2:1. Negative control samples where AAV production was prevented was achieved by transfecting the cells with only with ITR-GOI plasmid (i.e., Helper and Rep/Cap plasmids were omitted). For DBP western blot analysis of the results of the experiments, an anti-DBP polyclonal antibody from CusaBio was used (CSB-PA365892ZA01HIL).

B. Experimental Results

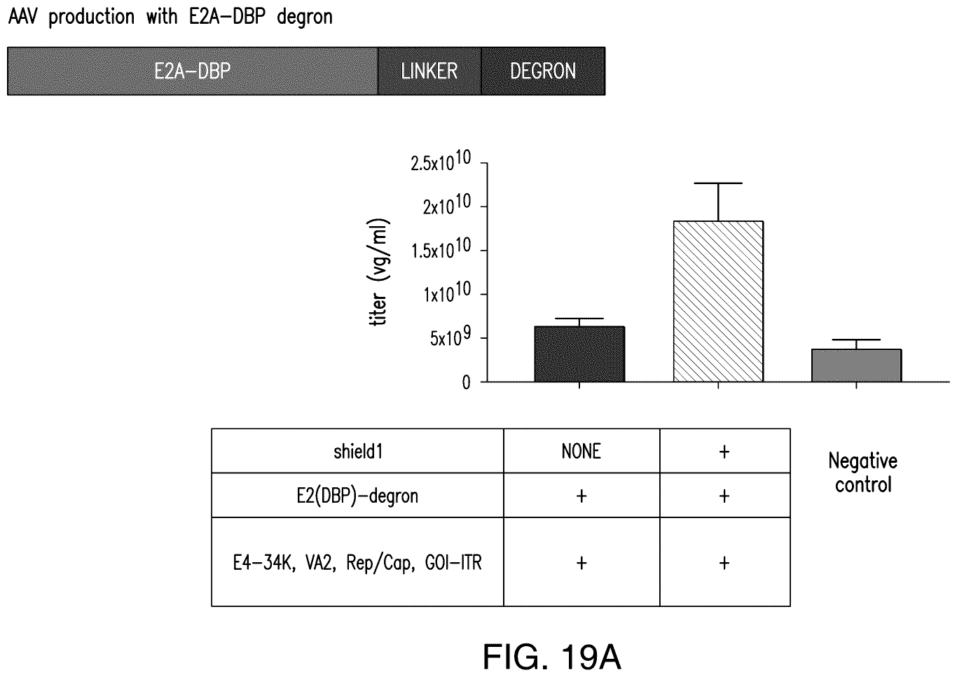

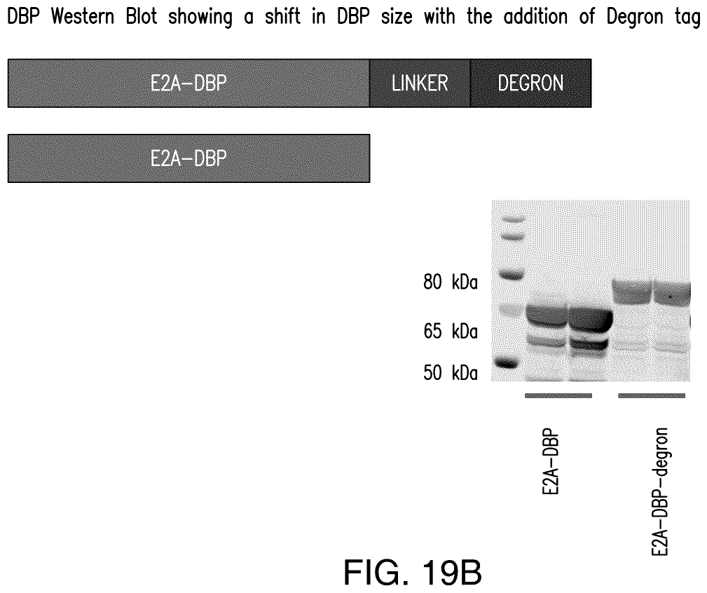

As indicated in FIG. 19A, DBP protein expressed from E2A gene was tagged with and FKBP derived degron motif. The plasmid expressing E2A-DBP-degron was transfected along with other plasmids expressing Rep/Cap, ITR-GOI, E4-E34K and VA2. At the end of 72 hours, the cells were lysed and AAV titer levels were analyzed. The cells transfected only with ITR-GOI plasmid (i.e., excluding Helper and Rep/Cap plasmids) were used as negative control samples. Without the addition of Shield1 molecule, the AAV production is observed at the same level as the background levels of the qPCR results of the negative control (solid bar on the left vs bar at the right). Upon the addition of Shield1 molecule the titer levels increased approximately 3-fold proving the regulation of AAV production using E2A-DBP with degron tag (striped middle bar).

FIG. 19B depicts a Western Blot highlighting the shift in the protein size of E2A-DBP protein due to the addition of the degron tag. The tagged protein is ˜12 kDa bigger than the untagged DBP protein. The two samples on the left are untagged DBP, while the samples on the right are for DBP with degron.

C. Sequences of Helper Genes Used

E2A-DBP nucleic acid sequence
ATGGCCAGTCGGGAAGAGGAGCAGCGCGAAACCACCCCCGAGCGC
GGACGCGGTGCGGCGCGACGTCCACCAACCATGGAGGACGTGTCGTCCCCGT
CGCCGTCGCCGCCGCCTCCCCGCGCGCCCCCAAAAAAGCGGCTGAGGCGGCG
TCTCGAGTCCGAGGACGAAGAAGACTCGTCACAAGATGCGCTGGTGCCGCGC
ACACCCAGCCCGCGGCCATCGACCTCGACGGCGGATTTGGCCATTGCGTCCAA
AAAGAAAAAGAAGCGCCCCTCTCCCAAGCCCGAGCGCCCGCCATCCCCAGAG
GTGATCGTGGACAGCGAGGAAGAAAGAGAAGATGTGGCGCTACAAATGGTGG
GTTTCAGCAACCCACCGGTGCTAATCAAGCACGGCAAGGGAGGTAAGCGCAC
GGTGCGGCGGCTGAATGAAGACGACCCAGTGGCGCGGGGTATGCGGACGCAA
GAGGAAAAGGAAGAGTCCAGTGAAGCGGAAAGTGAAAGCACGGTGATAAAC
CCGCTGAGCCTGCCGATCGTGTCTGCGTGGGAGAAGGGCATGGAGGCTGCGC
GCGCGTTGATGGACAAGTACCACGTGGATAACGATCTAAAGGCAAACTTCAA
GCTACTGCCTGACCAAGTGGAAGCTCTGGCGGCCGTATGCAAGACCTGGCTAA
ACGAGGAGCACCGCGGGTTGCAGCTGACCTTCACCAGCAACAAGACCTTTGTG
ACGATGATGGGGCGATTCCTGCAGGCGTACCTGCAGTCGTTTGCAGAGGTAAC
CTACAAGCACCACGAGCCCACGGGCTGCGCGTTGTGGCTGCACCGCTGCGCTG
AGATCGAAGGCGAGCTTAAGTGTCTACACGGGAGCATTATGATAAATAAGGA
GCACGTGATTGAAATGGATGTGACGAGCGAAAACGGGCAGCGCGCGCTGAAG
GAGCAGTCTAGCAAGGCCAAGATCGTGAAGAACCGGTGGGGCCGAAATGTGG
TGCAGATCTCCAACACCGACGCAAGGTGCTGCGTGCATGACGCGGCCTGTCCG
GCCAATCAGTTTTCCGGCAAGTCTTGCGGCATGTTCTTCTCTGAAGGCGCAAA
GGCTCAGGTGGCTTTTAAGCAGATCAAGGCTTTCATGCAGGCGCTGTATCCTA
ACGCCCAGACCGGGCACGGTCACCTTCTGATGCCACTACGGTGCGAGTGCAAC
TCAAAGCCTGGGCATGCACCCTTTTTGGGAAGGCAGCTACCAAAGTTGACTCC
GTTCGCCCTGAGCAACGCGGAGGACCTGGACGCGGATCTGATCTCCGACAAG
AGCGTGCTGGCCAGCGTGCACCACCCGGCGCTGATAGTGTTCCAGTGCTGCAA
CCCTGTGTATCGCAACTCGCGCGCGCAGGGCGGAGGCCCCAACTGCGACTTCA
AGATATCGGCGCCCGACCTGCTAAACGCGTTGGTGATGGTGCGCAGCCTGTGG
AGTGAAAACTTCACCGAGCTGCCGCGGATGGTTGTGCCTGAGTTTAAGTGGAG
CACTAAACACCAGTATCGCAACGTGTCCCTGCCAGTGGCGCATAGCGATGCGC
GGCAGAACCCCTTTGATTTTTAA
E2A-DBP amino acid Sequence
MASREEEQRETTPERGRGAARRPPTMEDVSSPSPSPPPPRAPPKKRLRR
RLESEDEEDSSQDALVPRTPSPRPSTSTADLAIASKKKKKRPSPKPERPPSPEVIVDS
EEEREDVALQMVGFSNPPVLIKHGKGGKRTVRRLNEDDPVARGMRTQEEKEESS
EAESESTVINPLSLPIVSAWEKGMEAARALMDKYHVDNDLKANFKLLPDQVEAL
AAVCKTWLNEEHRGLQLTFTSNKTFVTMMGRFLQAYLQSFAEVTYKHHEPTGCA
LWLHRCAEIEGELKCLHGSIMINKEHVIEMDVTSENGQRALKEQSSKAKIVKNRW
GRNVVQISNTDARCCVHDAACPANQFSGKSCGMFFSEGAKAQVAFKQIKAFMQA
LYPNAQTGHGHLLMPLRCECNSKPGHAPFLGRQLPKLTPFALSNAEDLDADLISD
KSVLASVHHPALIVFQCCNPVYRNSRAQGGGPNCDFKISAPDLLNALVMVRSLWS
ENFTELPRMVVPEFKWSTKHQYRNVSLPVAHSDARQNPFDF*
E4-34K (ORF6) Nucleic Acid Sequence
ATGACTACGTCCGGCGTTCCATTTGGCATGACACTACGACCAACAC
GATCTCGGTTGTCTCGGCGCACTCCGTACAGTAGGGATCGCCTACCTCCTTTTG
AGACAGAGACCCGCGCTACCATACTGGAGGATCATCCGCTGCTGCCCGAATGT
AACACTTTGACAATGCACAACGTGAGTTACGTGCGAGGTCTTCCCTGCAGTGT
GGGATTTACGCTGATTCAGGAATGGGTTGTTCCCTGGGATATGGTTCTGACGC
GGGAGGAGCTTGTAATCCTGAGGAAGTGTATGCACGTGTGCCTGTGTTGTGCC
AACATTGATATCATGACGAGCATGATGATCCATGGTTACGAGTCCTGGGCTCT
CCACTGTCATTGTTCCAGTCCCGGTTCCCTGCAGTGCATAGCCGGCGGGCAGG
TTTTGGCCAGCTGGTTTAGGATGGTGGTGGATGGCGCCATGTTTAATCAGAGG
TTTATATGGTACCGGGAGGTGGTGAATTACAACATGCCAAAAGAGGTAATGTT
TATGTCCAGCGTGTTTATGAGGGGTCGCCACTTAATCTACCTGCGCTTGTGGTA
TGATGGCCACGTGGGTTCTGTGGTCCCCGCCATGAGCTTTGGATACAGCGCCT
TGCACTGTGGGATTTTGAACAATATTGTGGTGCTGTGCTGCAGTTACTGTGCTG
ATTTAAGTGAGATCAGGGTGCGCTGCTGTGCCCGGAGGACAAGGCGTCTCATG
CTGCGGGCGGTGCGAATCATCGCTGAGGAGACCACTGCCATGTTGTATTCCTG
CAGGACGGAGCGGCGGCGGCAGCAGTTTATTCGCGCGCTGCTGCAGCACCAC
CGCCCTATCCTGATGCACGATTATGACTCTACCCCCATGTAG
E4-34K (ORF6) Amino Acid Sequence
MTTSGVPFGMTLRPTRSRLSRRTPYSRDRLPPFETETRATILEDHPLLPE
CNTLTMHNVSYVRGLPCSVGFTLIQEWVVPWDMVLTREELVILRKCMHVCLCCA
NIDIMTSMMIHGYESWALHCHCSSPGSLQCIAGGQVLASWFRMVVDGAMFNQRF
IWYREVVNYNMPKEVMFMSSVFMRGRHLIYLRLWYDGHVGSVVPAMSFGYSAL
HCGILNNIVVLCCSYCADLSEIRVRCCARRTRRLMLRAVRIIAEETTAMLYSCRTE
RRRQQFIRALLQHHRPILMHDYDSTPMCIFEQGGGGSGGGGSGGGGSMGVQVETI
SPGDGRTFPKRGQTCVVHYTGMLEDGKKVDSSRDRNKPFKFMLGKQEVIRGWEE
GVAQMSVGQRAKLTISPDYAYGATGHPGIIPPHATLVFDVELLKPE*
VA2 nucleic acid sequence:
ATGCTGTTTCCGGAGGAATTTGCAAGCGGGGTCTTGCATGACGGGG
AGGCAAACCCCCGTTCGCCGCAGTCCGGCCGGCCCGAGACTCGAACCGGGGG
TCCTGCGACTCAACCCTTGGAAAATAACCCTCCGGCTACAGGGAGCGAGCCAC
TTAATGCTTTCGCTTTCCAGCCTAACCGCTTACGCCGCGCGCGGCCAGTGGCC
AAAAAAGCTAGCGCAGCAGCCGCCGCGCCTGGAAGGAAGCCAAAAGGAGCG
CTCCCCCGTTGTCTGACGTCGCACACCTGGGTTCGACACGCGGGCGGTAACCG
CATGGATCACGGCGGACGGCCGGATCCGGGGTTCGAACCCCGGTCGTCCGCC
ATGATACCCTTGCGAATTTATCCACCAGACCACGGAAGAGTGCCCGCTTACAG
GCTCTCCTTTTGCACGGTCTAG
VA2 amino acid sequence:
MLFPEEFASGVLHDGEANPRSPQSGRPETRTGGPATQPLENNPPATGS
EPLNAFAFQPNRLRRARPVAKKASAAAAAPGRKPKGALPRCLTSHTWVRHAGGN
RMDHGGRPDPGFEPRSSAMIPLRIYPPDHGRVPAYRLSFCTV*
***

All publications, patents and other references cited herein are incorporated by reference in their entirety into the present disclosure

What is claimed is:

1. A method of regulating the production of recombinant adeno-associated virus (rAAV) vector particles, the method comprising:
a) introducing into a cell:
a. an rAAV comprising a gene of interest; and
b. a nucleic acid encoding a fusion protein, wherein the fusion protein comprises an AAV protein, and a degradation ligand-dependent degradation domain,
b) culturing the cell under conditions suitable for producing the rAAV vector particles; and
c) contacting the cell with a degradation ligand, wherein the degradation ligand binds to the degradation domain to regulate the expression of the AAV protein and thereby regulate the production of rAAV vector particles.

2. The method of claim 1, wherein the nucleic acid encodes a fusion protein, wherein the fusion protein comprises the AAV protein, a linker, and a degradation ligand-dependent degradation domain.

3. The method of claim 1 or 2, wherein the AAV protein is Cap.

4. The method of claim 1 or 2, wherein the AAV protein is a Helper protein.

5. The method of claim 4, wherein the Helper protein is E2.

6. The method of claim 1 or 2, wherein the AAV protein is Rep.

7. The method of claims 1-62, wherein the ligand-dependent degradation domain is derived from FKBP.

8. The method of claims 1-6, wherein the degradation ligand-dependent degradation domain is dihydrofolate reductase (DHFR).

9. The method of claims 1-6, wherein the degradation ligand-dependent degradation domain is an auxin induced degradation domain.

10. The method of any one of claims 1-9, wherein the degradation ligand is a small molecule ligand.

11. The method of claim 7, wherein the small molecule is Shield1.

12. The method of claim 8, wherein the small molecule is trimethoprim (TMP).

13. The method of claim 9, wherein the small molecule is auxin.

14. The method of any one of claims 1-13, wherein the cell is an E1a-expressing cell.

15. The method of claim 14, wherein the E1a-expressing cell is a HEK293 cell.

16. The method of any one of claims 6-15, wherein the Rep protein is Rep78, Rep68, Rep52, or Rep40 protein.

17. The method of any one of claims 6-16, wherein the degradation ligand-dependent degradation domain is fused to the C-terminal end of the Rep protein.

18. The method of any one of claims 6-16, wherein the degradation ligand-dependent degradation domain is fused to the N-terminal end of the Rep protein.

19. The method of claim 2, wherein the linker is a flexible linker.

20. The method of claim 2, wherein the linker is a rigid linker.

21. The method of any one of claims 1-20, comprising introducing into the cell a nucleic acid encoding a Cap protein.

22. The method of claim 21, wherein the nucleic acid encoding the fusion protein and the nucleic acid encoding a Cap protein, are introduced into the cell using at least one plasmid.

23. The method of claim 21, wherein the nucleic acid encoding the fusion protein and the nucleic acid encoding a Cap protein, are introduced into the cell using the same plasmid.

24. The method of claim 21, wherein the nucleic acid encoding the fusion protein and the nucleic acid encoding a Cap protein, are introduced into the cell using separate plasmids.

25. The method of any one of claims 1-24, wherein the rep, cap, or helper genes are under the control of a regulatory element.

26. The method of claim 25, wherein said regulatory element is a promoter.

27. The method of claim 25, wherein said regulatory element comprises a Tet response binding element.

28. The method of any one of claims 1-27, wherein the cell is a eukaryotic cell.

29. The method of claim 289, wherein the eukaryotic cell is an animal cell.

30. The method of claim 29, wherein the animal cell is a mammalian cell.

31. The method of claim 30, wherein the mammalian cell is a HEK cell.

32. The method of claim 30, wherein the mammalian cell is a Chinese Hamster Ovary cell.

33. A rAAV producing cell, wherein the cell comprises a nucleic acid encoding a fusion protein comprising an AAV protein, and a degradation ligand-dependent degradation domain.

34. The rAAV producing cell of claim 33, wherein the nucleic acid encoding a fusion protein comprising the AAV protein, a linker, and a degradation ligand-dependent degradation domain.

35. The rAAV producing cell of claim 33 or 34, wherein the AAV protein is selected from the group consisting of Rep, Cap, and Helper proteins.

36. The rAAV producing cell of claim 35, wherein the AAV protein is Cap.

37. The rAAV producing cell of claim 35, wherein the AAV protein is a Helper protein.

38. The rAAV producing cell of claim 35, wherein the AAV protein is Rep.

39. The rAAV producing cell of claims 33-38, wherein the degradation ligand is a small molecule ligand.

40. The rAAV producing cell of claims 33-39, wherein the degradation ligand-dependent degradation domain is derived from FKBP.

41. The rAAV producing cell of claims 33-39, wherein the degradation ligand-dependent degradation domain is dihydrofolate reductase (DHFR).

42. The rAAV producing cell of claims 33-39, wherein the degradation ligand-dependent degradation domain is an auxin induced degradation domain.

43. The rAAV producing cell of claim 40, wherein the small molecule is Shield1.

44. The rAAV producing cell of claim 41, wherein the small molecule is trimethoprim (TMP).

45. The rAAV producing cell of claim 42, wherein the small molecule is auxin.

46. The rAAV producing cell of any one of claims 33-45 wherein the cell is a eukaryotic cell.

47. The rAAV producing cell of 46, wherein the eukaryotic cell is an animal cell.

48. The rAAV producing cell of 47, wherein the animal cell is a mammalian cell.

49. The rAAV producing cell of 48, where the mammalian cell is a HEK cell.

50. The rAAV producing cell of 48, where the mammalian cell is a Chinese Hamster Ovary cell.

51. The rAAV producing cell of claim 33-45, wherein the cell is an E1a-expressing cell.

52. The rAAV producing cell of claim 51 wherein the E1a-expressing cell is a HEK293 cell.

53. The rAAV producing cell of any one of claims 38-45, wherein the ligand-dependent degradation domain is fused via the linker to the C-terminal end of the Rep protein.

54. The rAAV producing cell of any one of claims 38-45, wherein the cell is a mammalian cell and the ligand-dependent degradation domain is fused via the linker to the N-terminal end of the Rep protein.

55. The rAAV producing cell of claim 34, wherein the cell is a mammalian cell and the linker is a flexible linker.

56. The rAAV producing cell of claim 34, wherein the cell is a mammalian cell and the linker is a rigid linker.

57. The rAAV producing cell of claim 48-56, further comprising introducing into the cell a nucleic acid encoding a Cap protein.

58. The rAAV producing cell of claim 57, wherein the cell is a mammalian cell and the nucleic acid encoding the fusion protein and the nucleic acid encoding a Cap protein are introduced into the cell using at least one plasmid.

59. The rAAV producing cell of claim 57, wherein the cell is a mammalian cell and the nucleic acid encoding the fusion protein and the nucleic acid encoding a Cap protein, are introduced into the cell using the same plasmid.

60. The rAAV producing cell of claim 57, wherein the cell is a mammalian cell and the nucleic acid encoding the fusion protein and the nucleic acid encoding a Cap protein, are introduced into the cell using separate plasmids.

61. The rAAV producing cell of any one of claims 48-60, wherein the cell is a mammalian cell and the rep, cap, or helper genes are under the control of a regulatory element.

62. The rAAV producing cell of claim 61, wherein said regulatory element is a promoter.

63. The rAAV producing cell of claim 61, wherein said regulatory element comprises a Tet response binding element.

64. The method of any one of claims 1-32 or the rAAV producing cell of any one of claims 33-63 wherein two or more different AAV proteins are independently fused to degradation ligand-dependent degradation domains.

65. The method or rAAV producing cell of claim 64, wherein each different AAV protein is independently fused to a different degradation ligand-dependent degradation domain.


SEQUENCE LISTING (17 sequences)

SEQ ID NO: 1 - protein, 15 aa, synthetic construct
  REGION 1..15: note = Synthetic: flexible linker
GGGGSGGGGSGGGGS

SEQ ID NO: 2 - protein, 46 aa, synthetic construct
  REGION 1..46: note = Synthetic: rigid linker
AEAAAKEAAAKEAAAKEAAAKALEAEAAAKEAAAKEAAAKEAAAKA

SEQ ID NO: 3 - protein, 108 aa, synthetic construct
  REGION 1..108: note = Synthetic: amino acid sequence of the degron (FKBP), having a F36V and an L107P mutation
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 4 - protein, 159 aa, synthetic construct
  REGION 1..159: note = Synthetic: ecDHFR derived degron
MISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 5 - DNA, 480 bp, synthetic construct
  misc_feature 1..480: note = Synthetic: ecDHFR derived degron
  source 1..480: mol_type = other DNA
atgatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgggtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcggtaa

SEQ ID NO: 6 - protein, 148 aa, synthetic construct
  REGION 1..148: note = Synthetic: auxin derived degron
MGSVELNLRETELCLGLPGGDTVAPVTGNKRGFSETVDLKLNLNNEPANKEGSTTHDVVTFDSKEKSACPKDPAKPPAKAQVVGWPPVRSYRKNVMVSCQKSSGGPEAAAFVKVSLDGAPYLRKIDLRLYKSYDELSNALSNLFSSFT

SEQ ID NO: 7 - DNA, 447 bp, synthetic construct
  misc_feature 1..447: note = Synthetic: auxin derived degron
  source 1..447: mol_type = other DNA
atgggcagtgtcgagctgaatctgagggagactgagctgtgtcttggtcttcccggtggagatacagtggctccggtaaccggaaacaagagagggttctcagagacggttgatctgaagctaaatctgaataatgagcctgcaaacaaggaaggatctacgactcatgacgtagtgacttttgattccaaggagaagagtgcttgtcctaaagatccagccaaacctccggccaaggcacaagttgtgggatggccaccggtgagatcataccggaagaacgtgatggtttcctgccaaaaatcaagcggtggcccggaggcggcggcgttcgtgaaggtatcattagacggagcaccgtacttgaggaaaatcgatttgaggttatataaaagctacgatgagctttctaatgctttgtccaacttattcagctcttttacctga

SEQ ID NO: 8 - protein, 594 aa, synthetic construct
  REGION 1..594: note = Synthetic: amino acid sequence of the TIR-1 protein
MQKRIALSFPEEVLEHVFSFIQLDKDRNSVSLVCKSWYEIERWCRRKVFIGNCYAVSPATVIRRFPKVRSVELKGKPHFADFNLVPDGWGGYVYPWIEAMSSSYTWLEEIRLKRMVVTDDCLELIAKSFKNFKVLVLSSCEGFSTDGLAAIAATCRNLKELDLRESDVDDVSGHWLSHFPDTYTSLVSLNISCLASEVSFSALERLVTRCPNLKSLKLNRAVPLEKLATLLQRAPQLEELGTGGYTAEVRPDVYSGLSVALSGCKELRCLSGFWDAVPAYLPAVYSVCSRLTTLNLSYATVQSYDLVKLLCQCPKLQRLWVLDYIEDAGLEVLASTCKDLRELRVFPSEPFVMEPNVALTEQGLVSVSMGCPKLESVLYFCRQMTNAALITIARNRPNMTRFRLCIIEPKAPDYLTLEPLDIGFGAIVEHCKDLRRLSLSGLLTDKVFEYIGTYAKKMEMLSVAFAGDSDLGMHHVLSGCDSLRKLEIRDCPFGDKALLANASKLETMRSLWMSSCSVSFGACKLLGQKMPKLNVEVIDERGAPDSRPESCPVERVFIYRTVAGPRFDMPGFVWNMDQDSTMRFSRQIITTNGL

SEQ ID NO: 9 - DNA, 386 bp, synthetic construct
  misc_feature 1..386: note = Synthetic: DNA sequence of the TRE3G promoter
  source 1..386: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgggcgcgccaccgtcagatcgcctggagcaattccacaacacttttgtcttataccaactttccgtaccacttcctaccctcgtaaa

SEQ ID NO: 10 - protein, 248 aa, synthetic construct
  REGION 1..248: note = Synthetic: amino acid sequence of the transactivator protein
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 11 - DNA, 655 bp, synthetic construct
  misc_feature 1..655: note = Synthetic: DNA sequence of the CMV promoter
  source 1..655: mol_type = other DNA
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatt

SEQ ID NO: 12 - DNA, 1590 bp, synthetic construct
  misc_feature 1..1590: note = Synthetic: E2A-DBP nucleic acid sequence
  source 1..1590: mol_type = other DNA
atggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaa

SEQ ID NO: 13 - protein, 529 aa, synthetic construct
  REGION 1..529: note = Synthetic: E2A-DBP amino acid Sequence
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 14 - DNA, 885 bp, synthetic construct
  misc_feature 1..885: note = Synthetic: E4-34K (ORF6) Nucleic Acid Sequence
  source 1..885: mol_type = other DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtag

SEQ ID NO: 15 - protein, 422 aa, synthetic construct
  REGION 1..422: note = Synthetic: E4-34K (ORF6) Amino Acid Sequence
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPMCIFEQGGGGSGGGGSGGGGSMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 16 - DNA, 435 bp, synthetic construct
  misc_feature 1..435: note = Synthetic: VA2 nucleic acid sequence
  source 1..435: mol_type = other DNA
atgctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctag

SEQ ID NO: 17 - protein, 144 aa, synthetic construct
  REGION 1..144: note = Synthetic: VA2 amino acid sequence
MLFPEEFASGVLHDGEANPRSPQSGRPETRTGGPATQPLENNPPATGSEPLNAFAFQPNRLRRARPVAKKASAAAAAPGRKPKGALPRCLTSHTWVRHAGGNRMDHGGRPDPGFEPRSSAMIPLRIYPPDHGRVPAYRLSFCTV